(19) United States
(12) Patent Application Publication
(10) Pub. No.: US 2026/0137305 A1
(43) Pub. Date: May 21, 2026

(54) COMMUNICATION EXPRESSION USER INTERFACE

(71) Applicant: TEPY, Leichhardt (AU)

(72) Inventor: Jason Robert POTAS, Leichhardt (AU)

(21) Appl. No.: 19/427,529

(22) Filed: Dec. 19, 2025

Related U.S. Application Data
(63) Continuation of application No. PCT/AU2024/050658, filed on Jun. 21, 2024.

(30) Foreign Application Priority Data
Jun. 21, 2023 (AU) 2023901967

(51) Int. Cl. A61B 5/11; A61B 5/00; H10N 30/30

(52) U.S. Cl. CPC A61B 5/1126; A61B 5/686; A61B 5/7264; H10N 30/302; A61B 2562/164

(57) ABSTRACT

A human communication expression system comprises a user interface and a processor configured to receive sensor data from the user interface, process the received data to determine an intended communication, generate output data, and output the output data to the communication destination. The processor comprises a data input interface, a communication processor, and a data output interface. The communication processor is configured to process the received sensor data to determine the intended communication represented by the sensor data. The user interface comprises at least one sensor module configured to sense communication expressions of a user and output a sensor data signal, and a support structure adapted to be worn by the user. The support structure is configured to hold the at least one sensor module relative to the user's body so that the at least one sensor module senses the communication expressions of the user.

### CROSS-REFERENCE TO RELATED APPLICATIONS

The present application claims priority from Australian Provisional Patent Application No 2023901967 filed on 21 Jun. 2023, the content of which is incorporated herein by reference.

### TECHNICAL FIELD

The present disclosure broadly relates to human-machine interfaces and, more particularly, to a system for, and a method of, human expression sensing and communication.

### BACKGROUND

Humans interface with devices by communicating their thoughts and ideas. The most natural way to communicate complex ideas and thoughts is with language, usually in the form of speech, but language can also be expressed using other non-verbal approaches like sign language, writing, and typing.

Humans interface with technology through a variety of input devices, including keyboards, mouses, game controllers (e.g. buttons and joysticks), touchscreens, and voice recognition software as examples. Input devices are an essential part of how humans interface with technology, allowing them to input commands, interact with graphical interfaces, control devices and software applications (apps), and communicate with others through devices.

A good interface between technology and humans is one that is intuitive and natural to use. By far the most common way for human users to interface with technology is the use of keyboards (on a touchscreen or otherwise), and typing is perhaps the most common way that humans interface with technology. The use of voice recognition software permits hands-free communication of thoughts to the device interface. The drawback from using voice recognition is that the user must broadcast their thought or message, such that nearby bystanders can hear it; in addition, voice recognition becomes less accurate as the ambient noise increases and interferes with the sound being decoded into speech. The advantage of using tactile interfaces, such as keyboard and touch screens, is that it is private (messages are communicated without broadcasting to bystanders), but it is a much slower form of communication, and is not overly eyes-free and hands-free as it requires considerable engagement by the user to execute it.

Any discussion of the background art throughout the specification should in no way be considered as an admission that such art is widely known or forms part of common general knowledge in the field.

### SUMMARY

The present disclosure relates to sensors and a wearable device for silent (and/or private), voice-free, hands-free and/or eyes-free communication to input devices, and has broad applications related to voice recognition, keyboards and/or touch screens for interfacing with devices and/or digital technologies. Some embodiments are particularly adapted for extracting information from the human body regarding intended communication.

Described herein are methods, systems, and apparatus for the interpretation and utilization of intended communications from a human subject, specifically where the intended communications are associated with the interpretation of a thought that is intended to be communicated. The technologies convert physical changes of communication-expressing structures (CES) into electrical signals using one or more sensors. These electrical signals are then transformed into data representative of the intended communications using a language processor in the form of a processing unit or processor equipped with machine learning algorithms and language models. The data may then then be utilized to perform actions or generate outputs corresponding to the intended communications. Also described are methods of separating the interpreted input into conversation components and command components, facilitating both human-machine interaction and software application and/or device control. The methods and systems described find utility in silent communication, aiding individuals with disabilities or unique communication needs, providing an alternative interface for visually impaired individuals, communication in noisy environments, private or stealth communication, language translation, and/or replacing traditional input interfaces in electronic devices. It enables user interaction with digital technology and provides an intuitive human-device interface.

A Silent Language Interface (SLI) permits simultaneous eyes-free, hands-free and voice-free, private (silent) communication. Communicating intended thoughts using language silently, and without the use of eyes and hands mimics the experience of telepathy. “Silent” means that the communication expression does not need to be audible. However, it will be understood that the communication expression need not be silent.

As used herein, references to the user's thoughts, and body signals associated with the user's thoughts, generally refer to brain activity associated with an intention to communicate and an intended communication. The communication may be for the purpose of sharing information, for example in the form of a message to another person. The communication may also be a command, for example for the purpose of effecting an action via technology.

A SLI is possible because intended communications are able to be expressed silently by human beings, and eyes- and hands-free communication of intended thoughts by the user is possible in a format that can be transmitted and interpreted digitally. This permits the user to send their intended communications and ideas as a message to a device, as naturally as possible, without broadcasting their intended communications to the surroundings.

Some existing methods try to decode electrical activity of the brain from electroencephalograms (EEG) through electrodes placed on the scalp to extract the meaning. However, these signals are highly complex, and the compound nature of EEG signals do not permit decoding of intended communications with much accuracy or resolution. Some existing methods aim to decode brain signals to achieve a SLI using more invasive means with greater success, however, this approach requires brain surgery, and bring numerous drawbacks which may include:
1) It requires implanting sensors in the brain which is invasive and poorly reversible.
2) The signals for decoding intended communications are acquired from brain signals which are unrefined and difficult to decode. It therefore relies on large computational resources to decode intended communications into a form that can be received by a device.
3) Signals from higher brain regions are difficult to decode and to generalize the extracted information across different users, so the device needs to be trained for each individual user.
4) Precise sensor implant positioning is difficult to reproduce in multiple subjects. This adds further difficulty for decoding and generalisation across different users, and therefore contributes to the need to train each user for their unique sensor location.
5) The process of generating usable signals from the brain is largely unnatural, so the user is required to learn how to use the system in order to convey their intended communications. For example, they may be required to imagine handwriting individual characters to spell out their message, which, unlike writing normally, is a cognitively challenging exercise.
6) The significant cognitive load required to use a SLI makes it difficult for the user to multi-task. Thus, while signals decoded from the brain is hands free, the user may be too engaged cognitively to then make use of their free hands.
7) The volume of information extracted from a user's brain is very large and therefore requires significant computational resources, so using a SLI that operates on invasive brain signals is expensive.
8) The number of users who would subject themselves to brain surgery is low, therefore generating large data sets representing many users is difficult and a slow process.

One prevalent approach in existing technologies to overcome these problems with SLIs that operate on brain structures is to capture signals that represent thought expression from the peripheral body structures. For this, a peripheral Silent Language Interface (PSLI) may be used. The main approach of applying PSLIs involves the sensing of electromyography (EMG) signals. EMG-based peripheral interfaces work by detecting and decoding the electrical signals generated by muscle fibres during contraction. For example, during speech, EMG can be used to capture the muscle activity involved in speech articulation, which can subsequently be used to translate a part, or all, of the intended communication. However, the primary issue with current EMG-based methods, is that the signal readings lack reproducibility, as they primarily rely on surface electrodes, which are prone to signal instability.

Some prior art methods record electroneurography (ENG) signals rather than EMG signals from the surface electrodes, which are signals arise from nerves rather than muscle cells. ENG recording presents with the same limitations as EMG, however the signals are orders of magnitude smaller than EMG, so the problems with recording stability are even greater for ENG.

The methods described herein provide alternative approaches for PSLIs that overcome the problems arising from standard EMG approaches. Furthermore, applications of using this technology for communication and controlling devices and apps is described.

The present disclosure pertains to methods, systems, and devices for interpreting and utilizing intended communications from a subject, with the thought-interpretation method implemented (or implementable) in a digital device.

The method comprises the conversion of physical or electrical changes in communication-expressing structures (CES), such as facial muscles, articulatory organs, and/or body surface areas on the arms, legs, hands, shoulders, etc., into a usable and processable form via one or more transducers. The sensor systems described herein may be adapted to sense electrical changes in the user's body, for example subcutaneously, without discernible or visible movement of the user's body, so that those electrical changes (which may be precursors to movement, for example a tensing of muscles) are interpreted as communication expressions.

For example, body movements or other communication expressions may be represented by electrical signals via the use of sensors configured to monitor and detect such expressions. These sensors may be of various types including piezoelectric, piezoresistive, capacitive, resistive, inductive, force-transductive, magnetoresistive, optical and/or electrodes. Combinations of different transducers and/or sensors may be used, for example to sense different types of movement, suggestions, or other communication expressions conveyed by the human body. The sensors may be configured to extract specific features from the CES, such as electrical activity, movement, positioning, and/or to extract distance information from a reference point to one or more CES.

In the context of the system, the sensors may be integrated into a module, housing, scaffold, bracket, or fabric that is in direct or indirect contact with the CES or positioned in close proximity with or without contact. This system setup enables the effective and accurate gathering of the physical and or electrical changes of CES for the thought interpretation process.

In some embodiments the thought-interpretation device may serve as the primary apparatus, containing the sensors, one or more processing units, and interface and/or output modules. The sensors capture the physical and/or electrical changes of the CES being monitored, and convert them into electrical signals that may be subsequently digitised. The processing unit uses machine learning algorithms such as neural networks and language models to transform these electrical signals into data representative of intended communications. To improve accuracy, the processing may include filtering to separate intended communication signals from other signals, such as unrelated movement artifacts.

The resulting data is used by the interface and/or output module to perform actions or generate outputs that correlate with the intended communications. This includes, but is not limited to, silent communication, device or software application control, enabling people with disabilities or unique communication needs to convey their thoughts, and communication in noisy environments or during language translation. It may also be used for communication, translation, as well as many other applications.

Additionally, the methods described enable the division of interpreted input into conversation components and command components. Conversation components are aimed at facilitating human-human or human-AI interactions and can be transformed into synthesized speech or text, while command components can be translated into control commands for interfacing with other digital technologies.

Overall, methods and systems described herein present an efficient and intuitive way to interpret and use intended thought communications from a subject, offering a novel human-digital interface that simplifies communication with other humans and digital technologies. It opens new avenues in digital technology interaction, replacing traditional methods of control such as keyboards, touchscreens, or voice recognition.

In one aspect there is provided a user interface for sensing human communication expressions, the user interface comprising: at least one sensor module configured to sense communication expressions of a user and output a sensor data signal; and a support structure adapted to be worn by a user, and configured to hold the at least one sensor module relative to the user's body so that the at least one sensor module senses the communication expressions of the user.

The at least one sensor module may comprise: a first sensor component having one or more sensor elements; and a second sensor component having one or more reciprocating components for activating the one or more sensor elements; and the first sensor component and the second sensor component may be positioned relative to one another in the sensor module so that communicating movements of the user cause the reciprocating components to move relative to their respective sensor elements thereby causing the sensor elements to sense the communicating movements of the user.

The one or more sensor elements may comprise piezoelectric sensors.

The first sensor component may comprise an interface component configured to guide a relative position and relative movement between the first sensor component and the second sensor component, and when a user's communication expressions affect the at least one sensor module, the interface component causes relative movement between the first sensor component and the second sensor component in a manner so that the one or more reciprocating components activate the one or more sensor elements.

The at least one sensor module may comprise a housing, and the interface component moves against the housing when the user performs a communication expression, causing the sensor elements to distort.

The second sensor component may comprise a cantilever that abuts the user and transfers the user's movement to the at least one sensor module by causing the second sensor component to move relative to the first sensor component when the user moves, thereby enhancing a directional sensitivity of the at least one sensor module.

The at least one sensor module may be attached to the support structure at a reference point, and movement of the user may be sensed relative to the reference point.

The at least one sensor module may comprise a pair of sensor modules positioned relative to one another and wherein sensor signals from the pair of sensor modules are combined to amplify the sensor data signal.

The at least one sensor module may comprise a combination of two or more sensor types selected from the group comprising: piezoelectric sensors, optical sensors, electromyography sensors, biopotential sensors, strain gauge sensors, load cells, force-sensitive resistors, force transducers, capacitive sensors, resistive sensors, inductive sensors, magneto resistive sensors, and acoustic sensors.

The at least one sensor module may comprise a flexible and/or elastic fabric.

The user interface may comprise two sensor modules held relative to one another via a flexible and/or elastic fabric, wherein the two sensor modules are configured to sense relative positions of the two sensor modules relative to one another, wherein the relative positions are indicative of the user's movement.

The support structure may be configured to hold the at least one sensor module relative to a speech articulator of the user.

The support structure may be configured to hold a first sensor module and a second sensor module to be oriented substantially orthogonal relative to one another.

The support structure may be configured to hold the at least one sensor module perpendicular to a communication expression structure of the user.

The at least one sensor module may comprise at least one subcutaneous part. The at least one sensor module may be configured to be subcutaneously applicable.

In another aspect there is provided a human communication expression system comprising: a user interface as described; and a processor configured to: receive sensor data from the user interface; process the received data to determine an intended communication; generate output data; and output the output data to the communication destination.

The processor may comprise: a data input interface; a communication processor; and a data output interface, wherein the communication processor is configured to process the received sensor data to determine the intended communication represented by the sensor data.

In one aspect, the present disclosure provides a speech-interface system that includes a proximal articulator module and a base module coupled by a communication link. The proximal articulator module is configured to be located at or adjacent to one or more speech articulators of a user and comprises an articulator-proximate sensing assembly arranged to obtain articulator-derived signals from one or more communication-expressing structures, including at least facial, perioral, mandibular, craniofacial or oral tissues. The articulator-proximate sensing assembly includes one or more articulator sensors or sensor arrays configured to detect articulatory movements associated with linguistic articulatory expressions of the user and, in response, to generate articulator-derived signals representing those articulatory movements. The base module comprises at least one processor and a communication interface, and is communicatively coupled to the proximal articulator module so that the articulator-derived signals are provided to the base module for processing. The processor is configured to process, or to cause one or more remote computing resources to process, the articulator-derived signals to generate linguistic output representing an utterance of the user and to provide the linguistic output to an output device for presentation as human-perceptible communication, such as synthesized speech and/or text. This architecture supports natural, eyes-free, hands-free and voice-free communication by exploiting silent or sub-audible speech articulations at the level of peripheral communication-expressing structures rather than relying on airborne acoustic signals.

In some embodiments, the articulator sensors comprise one or more of biomechanical deformation sensors, piezoelectric or strain sensors, capacitive or optical sensors, depth-sensing components, and electromyographic sensors configured to sense muscle activity associated with the speech articulators. The proximal articulator module and the base module may cooperate such that the proximal articulator module primarily performs signal acquisition, while the base module performs linguistic decoding of the articulator-derived signals using the at least one processor. The processor can map patterns in the articulator-derived signals to linguistic units such as phonemes, visemes, syllables, words, phrases or sentences, assemble those units into linguistic output and, in some embodiments, generate both a communication component and a command component from the same decoded communication. The communication component represents text or speech content intended for a human recipient and may be rendered as text and/or synthesized speech, while the command component represents a control intent that can be used to control other devices, software applications or automated assistant services. This allows a single silent linguistic expression to serve both as conversational content and as a control signal, increasing efficiency and enabling rich interaction with digital systems without audible speech.

In another aspect, the disclosure provides a speech-interface system in which the sensing assembly comprises one or more depth-sensing components, such as time-of-flight depth sensors or sensor arrays, LiDAR sensors or sensor arrays, structured-light depth cameras, infrared depth cameras, stereo depth cameras, thermal depth cameras, or combinations thereof. The one or more depth-sensing components may comprise depth sensors, depth sensor arrays, or multiple spatially distributed depth sensor arrays arranged so that their field of view extends to both intra-oral articulators within an oral cavity of the user and perioral articulators external to the oral cavity. The depth-sensing components are configured to generate depth data representing distances between the depth-sensing components and one or more speech articulators during linguistic articulatory expressions, and the processor is configured to process the depth data to decode those expressions and generate linguistic output corresponding to an intended utterance of the user. In some implementations the linguistic articulatory expressions comprise silent or sub-audible speech performed without reliance on airborne acoustic signals generated by vibrating vocal folds. This depth-based configuration enables non-contact or low-contact sensing of detailed articulator motion, improving comfort and hygiene and reducing mechanical loading on the tissues while still providing precise, high-resolution information about intra-oral and perioral movements.

In a further aspect, the disclosure provides a proximal articulator module for a speech-interface platform. The proximal articulator module includes an anatomically conforming, adjustable mounting structure configured to be worn on or supported by a head or face region of a user and to support a sensor assembly. The sensor assembly may be positioned at or adjacent to one or more speech articulators via one or more support arms, extensions, or intermediate support structures, and arranged to maintain a defined spatial registration with one or more facial, perioral, mandibular, craniofacial or intra-oral structures. An articulator-proximate sensing assembly supported by, or electrically coupled via, the mounting structure comprises one or more articulator sensors or sensor arrays configured to capture, from the one or more speech articulators, mechanical movements and/or electrical signals associated with muscle activity and, in response, to generate articulator-derived signals representing articulatory movements associated with linguistic articulatory expressions. A module interface is configured to convey these articulator-derived signals to a local or remote base module for processing. This proximal articulator module enables robust and repeatable alignment of sensors to underlying articulators, supports per-user calibration while maintaining comfort, and permits the sensing assembly to be used with a variety of communication back-ends and processing architectures.

In another aspect, the disclosure provides a speech-interface system including one or more subdermal articulator sensors implanted at or adjacent to one or more speech articulators of a user and configured to capture electrical signals associated with muscle activity during linguistic articulatory expressions. At least one subdermal conductive pathway may be permanently implanted beneath the user's skin and electrically connected to the subdermal articulator sensors and to one or more subdermal presenting electrodes located beneath the skin at a region adjacent a mounting location for a base module. The base module includes at least one processor and an electrical coupling interface with one or more external electrodes configured, in use, to be positioned on the skin adjacent the subdermal presenting electrodes so as to receive articulator-derived signals via biopotentials measured across the external and subdermal electrodes. The processor is configured to process the articulator-derived signals to generate linguistic output representing an intended utterance of the user. This implanted-sensor configuration provides a highly stable, low-noise signal path that can be used for long-term or continuous silent communication, while allowing the external base module to be attached and detached without disturbing the implanted components and leaving minimal visible hardware on the user.

The disclosure also encompasses methods and computer-readable media for operating the speech-interface platform. In one method, an articulator-proximate sensing assembly of a proximal articulator module worn at or adjacent to speech articulators obtains articulator-derived signals indicative of articulatory movements corresponding to linguistic articulatory expressions. The articulator-derived signals are communicated from the proximal articulator module to a base module comprising at least one processor via a communication link that may include a wired connection, a wireless connection, or indirect electrical coupling through implanted conductive structures. The processor decodes linguistic units representing an intended utterance of the user, generates linguistic output based on those units, and causes an output device to present human-perceptible communication based on the linguistic output. Instructions stored on a non-transitory computer-readable medium may, when executed by the processor of the base module or by cooperating remote computing resources, cause these steps to be performed. These method and medium aspects provide implementation flexibility across on-device and cloud-based processing environments, enabling the same articulator-sensing hardware to be used with evolving decoding models and services without changing the physical interface to the user.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

FIG. 1A is a representation of an embodiment of a human movement communication system.

FIG. 1B is a representation of another embodiment of a human movement communication system.

FIG. 2 is a schematic representation of the electrical behaviour of piezoelectric crystals.

FIG. 3A is a schematic representation of an arrangement of piezoelectric sensor elements to extract information about movement of a surface.

FIG. 3B is a schematic representation of an arrangement of a piezoelectric sensor element to determine contraction and stretch of a surface.

FIG. 4A is a schematic representation of piezoelectric sensor elements arranged to detect slip.

FIG. 4B is a graphical representation of a measured signal including a microslip.

FIG. 5 is a schematic representation of an assembly of piezoelectric sensor elements inside a sensor housing that can sense movements in 2 dimensions and with direction selectivity.

FIG. 6 is a schematic representation of an example of how a sensor can be used to resolve movement in 3 dimensions.

FIG. 7 is a schematic representation of an assembly for measuring opposing signals that can be used to augment the signals response, thereby increasing sensor sensitivity.

FIG. 8 shows example embodiments of sensor support structures and illustrates how to hold sensors onto the face using sensor support structures.

FIG. 9 shows an embodiment of an elasticated fabric sensor support.

FIG. 10 shows various example embodiments of piezoelectric sensing devices tuned for direction selectivity of movement using non-uniform elasticated fabric.

FIG. 11 shows example embodiments of sensor devices using elastic straps, and by combining a rigid holder such as a chin cup, with elastic straps.

FIG. 12 shows how Time-of-Flight information can be used to determine different communication-expressing states.

FIG. 13 shows how binocular imaging can be used to extract 3-dimensional information from the body.

FIG. 14 shows how EMG signals can be channeled under the skin, using electro-conducting tattoos or small wires for permanent signal acquisition solutions and reducing the need for skin electrodes.

FIG. 15A is a flow diagram of an embodiment of a method of transforming thought expressing signals into part or complete thoughts.

FIG. 15B shows how signals from sensors can be extracted and associated with components of intended communications.

FIG. 16 shows how to make an AI-interface from a large language model that can separate device control instructions from conversational content that was extracted and processed from intended communications.

### DETAILED DESCRIPTION

Peripheral Silent Language Interfaces (PSLIs) are an innovative technology facilitating the interface between humans and devices.

Peripheral here refers to the placement or position of at least some parts of the interface relative to a structure that lies outside the central nervous system (i.e. outside brain and spinal cord).

Unlike conventional methods of interaction that rely on explicit commands delivered through speech, text or gestures, PSLIs deciphers biological signals from the body that have been acquired from more unconventional means. This interface is achieved by extracting and deciphering biological signals that were initiated from the brain and transformed along the efferent pathway before affecting the end organs. These biological signals, representing the user's intended communications, are intercepted and decoded prior to the end organ modifying the external environment. Thus, the key feature of PSLI lies in its ability to capture and decode biological signals from the body before they engage with the final end organs, and interpreting them to estimate or determine the user's intent.

In an embodiment, a PSLI may be applied to interpret silent speech. Here, biological signals may be extracted from the movement of lips and surrounding biological structures, and deciphered without engagement of the vocal cords. This enables users to express their communication thoughts silently, providing a means of communication that does not necessitate verbalization or physical interaction with a device in the same way as holding a smartphone, looking at the screen and texting on a touch screen.

As used herein, a PSLI may also be used to extract intended communications from sign language, for example where the recipient of the communication does not normally understand sign language. Thus, in such an embodiment, the PSLI extracts and decodes signals from the muscles in the arms and/or hands prior to, or as they modify the position of the arms and/or hands. The user is then able to convey a message to a recipient party through gestures made with one or more body parts, without the requirement for the recipient to understand sign language.

A PSLI functions by inferring the user's intention based on the extracted biological signals, leveraging signal processing and machine learning techniques.

This technology marks a shift from conventional human-device interfaces, departing from traditional forms, and enabling an intuitive and seamless interaction. PSLIs allows for communication that bypasses the need for the user's voice, eyes, and in most cases hands (i.e. except for sign language applications as described herein), thereby providing a more direct link between intention and technological response. As such, PSLIs have wide-ranging potential applications in improving and diversifying the ways in which users can interface with technology.

The present disclosure relates to the sensors required for the extraction of information from soundless signals such as mechanical, optical, or electrical changes, that arise from the body and that represent an intended communication.

### System Overview

Described herein is a system that includes PSLI sensors. The PSLI sensors are designed to capture biosignals for translation into discernible communication. Primarily, these sensors overcome the prior-art shortcoming of poorly reproducing electrical sensing from EMG signals acquired from skin electrodes. Three key approaches are described here: mechanical, optical sensing, and a modified electrical approach that reduces reliance on skin electrodes. The innovation may use one or a combination of these sensor types and approaches for capturing biosignals resulting from a person's intended communication expression.

Mechanical sensing is achieved by capturing surface distortions on the body related to intended communication expressions. This includes detecting physical changes of communication expressing structures (CES) that occur during intended communication expression as a person sets an intention (i.e., as the person decides what they are going to communicate). These physical changes may be observed either directly from the CES itself, or indirectly from a fabric or structure that is in direct contact with, or indirect contact with, or in proximity to the CES.

Optical sensing is achieved by capturing optical information of CES as they undergo physical changes. This includes the intricate details of how the lips, tongue, and other related elements around the oral cavity move during intended communication expression, such as silent lip movements.

One or more of the biosignals captured by the sensors, or a combination of the sensor types, are subsequently used to decode and/or translate the intended communication(s) from one or more CES. As such, a PSLI system with these sensors can be used to substitute traditional user interfaces, such as keyboards, touchscreens and speech recognition software.

The technology has a variety of applications including permitting silent and stealth communication, communication in noisy environments, language translation, various assistive technology applications, permit communication in situations that otherwise obstructs speech (e.g. wearing breathing apparatus), and device, software application (app), and artificial intelligent system interfacing and control.

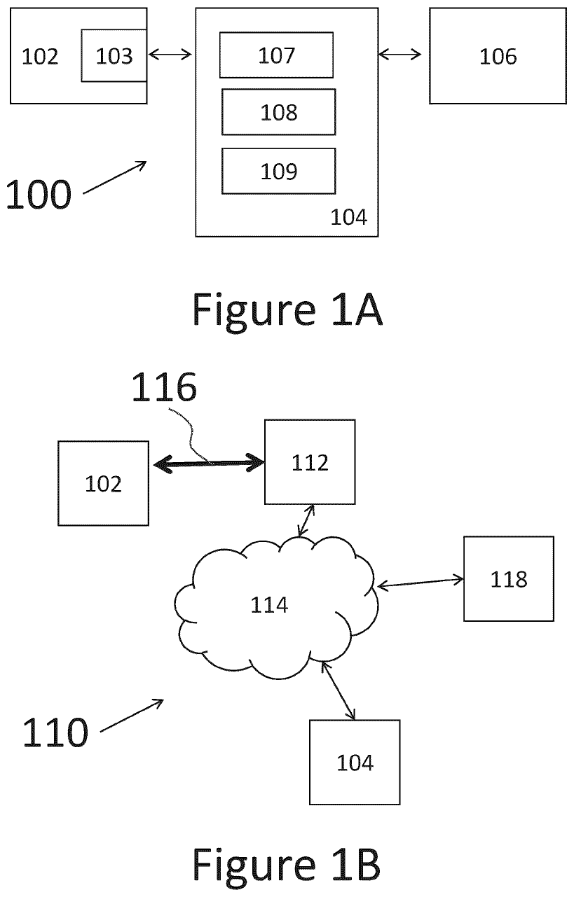

FIG. 1A of the drawings shows an embodiment of a human expression communication system 100 comprising a sensor user interface 102 in communication with a processor 104 configured to receive sensor data from the sensor user interface 102, process the received data to determine a communication destination, to generate output data, and to output the output data to the communication destination. The output data may be output to the communication destination via an output interface 106, for example in the form of a software interfacing module comprising a driver, application programming interface (API), or similar. The processor 104 comprises a data input interface 107, a language processor 108, and a data output interface 109. The language processor 108 is configured to process the received sensor data. For example, in some embodiments the language processor 108 may be configured to determine the communication destination, the communication intent, and/or the communication content, by determining a meaning of a communication represented by the sensor data.

The processor is configured to: receive sensor data from the user interface; process the received data to determine an intended communication; generate output data; and output the output data to the communication destination.

In embodiments the processor comprises: a data input interface; a communication processor; and a data output interface, wherein the communication processor is configured to process the received sensor data to determine the intended communication represented by the sensor data.

The sensor user interface 102 comprises one or more sensor assemblies and/or sensor modules as described elsewhere herein, and a sensor communication interface 103. The processor 104 may be in the form of a smartphone, laptop, desktop, or other similar computing device. The language processor 108 may include one or more processing modules, for example a machine learning (ML) module, a large language model (LLM) module, or other data and/or language processing modules.

In some embodiments, the user interface 102 may comprise the processor 104, for example where the sensor modules and the processor are collocated and held together by the support structure.

The communication destination may be a digital device like a drone, for example where a control command is provided, or the communication destination may be a communication device like a smartphone, for example where a text or voice message is sent. In some embodiments the communication destination may be predetermined or predefined, so that interpretation of the message involves interpreting the content of the message to be sent to the predetermined destination.

In some embodiments, determining the communication destination involves determining a communication intent, for example whether the communication is a command for controlling a device, whether the communication is a message to be sent to a recipient, whether the communication is a voice or text to be sent via a mobile carrier, etc. In some embodiments the destination may be the user interface itself, for example an audio output at the user interface based on the sensed communication expression.

FIG. 1B of the drawings shows another embodiment of a human expression communication system 110. In this embodiment, the sensor user interface 102 is in communication with the processor 104 via a relaying device 112 and a communication network 114. For example, the sensor user interface 102 sends sensed sensor data to a relaying device 112 (being in the form of a smart phone, tablet or laptop) via wireless via Bluetooth or Wi-Fi, or wired via a USB connection 116. The relaying device may then relay the sensed data to the processor 104 (e.g. a server, for example in the form of a cloud-based server) via a mobile network and/or a data network 114 such as the Internet. The processor 104 is configured to process and interpret the received sensor data and, based on the processing and interpreting, transmit a communication to a recipient device 118.

The sensor communication interface 103 may be a wired or wireless communication interface, allowing the sensor device 102 to transmit the sensor data to the relaying device 112 and/or the processor 104.

In some embodiments the human expression communication system may also contain the sensor-human interface 102 along with the processor 104, which may communicate directly to a recipient or device 118 via an interface 103.

### Sensor Devices

Language is the most natural way for humans to articulate their thoughts to the outside world. The methods described herein decode human thoughts as intended communications from the activation of CES during language expression. The user silently expresses what they wish to communicate, for example by moving their lips, arms and/or hands, while sensors extract the biosignals from the body generated by the intentional movements and the system translates these into part or complete intended communications using pattern recognition of the captured signals.

The disclosed technology can be embodied in various forms of sensing devices or modules. These sensing devices can be affixed directly onto the surface of a user's body, attached to wearable materials such as fabric-based articles, or secured to non-flexible or rigid structures or structures with flexible and rigid components, in proximity to the user's body, such as via supporting fabrics, scaffolds, brackets, or housing structures. A more permanent solution is achievable by implanting the sensors under the skin. Any of these sensing devices are configured to capture and analyse biological signals representing intended communications associated with intended actions and/or intended communication that were generated by the brain and propagated through the body and affect their target end organs.

The attachment of the sensing components may be accomplished through a variety of mechanisms, including but not limited to adhesive means, mechanical fastening, magnetic coupling, or through integration into articles worn by the user. The sensing devices may be standalone modules or they can be incorporated into, or used in conjunction with, other technologies or devices, such as wearable technologies, clothing, prosthetics, or any other items located in proximity to, or on the body.

One embodiment of a PSLI device is a wireless wearable technology that holds one or more of the described sensors and communicates these signals wirelessly to a processor where it can undergo adequate interpretation.

It should be understood that the abovementioned configurations for the sensing devices and attachment methods are exemplary and not limiting, and the sensing devices can be affixed to or integrated within any structure or item that facilitates the capture of relevant biological signals. Other forms and embodiments are also within the scope of this description.

Consider examples where the CES being sensed is the lip movements from silently mimed speech or from movements on the forearm during sign language. There are a variety of possible variations to how the sensors can be configured for capturing different features from such thought expressions. Distortions that occur on one or more CES may be detected mechanically, optically, or electrically, and/or via other suitable means.

FIG. 1 shows an example of a mechanical sensor, which may be a piezoelectric crystal that is distorted by the movement of a CES as the user expresses their intended communications. A Piezoelectric crystal in a steady state shows little or no potential difference between its two opposite surfaces 211, 213. When the piezoelectric crystal is acutely bent (concave 215, or convex 217) a voltage across the two surfaces can be measured. Depending on the direction of the bend, the voltage may be positive 215 or negative 217. Throughout the examples, the voltmeter 219 has the “+” connected to the piezo surface 211 and the “−” to the conducting plate 213 on the opposite surface to ensure distortions in the opposite directions will ensure opposite voltages.

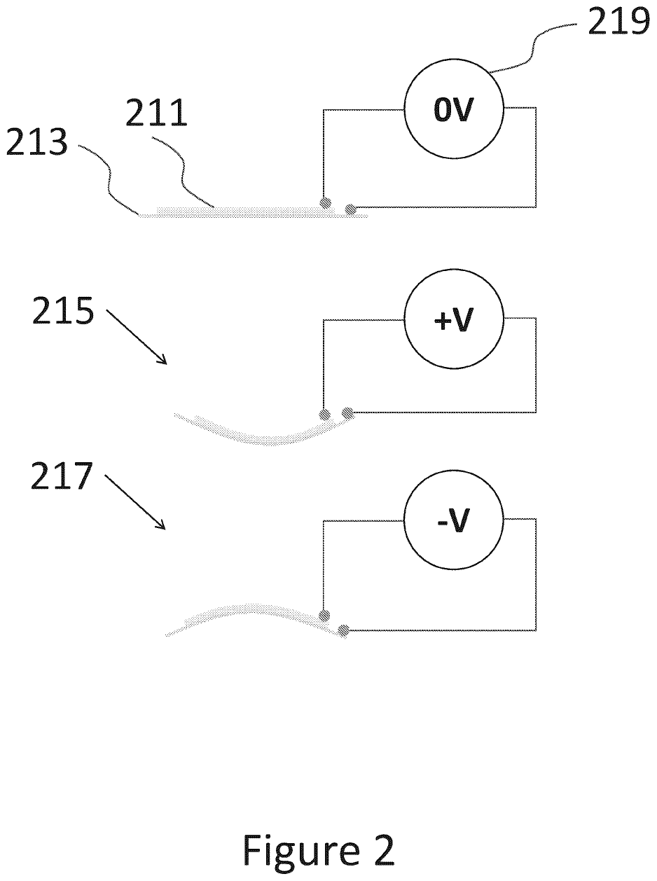

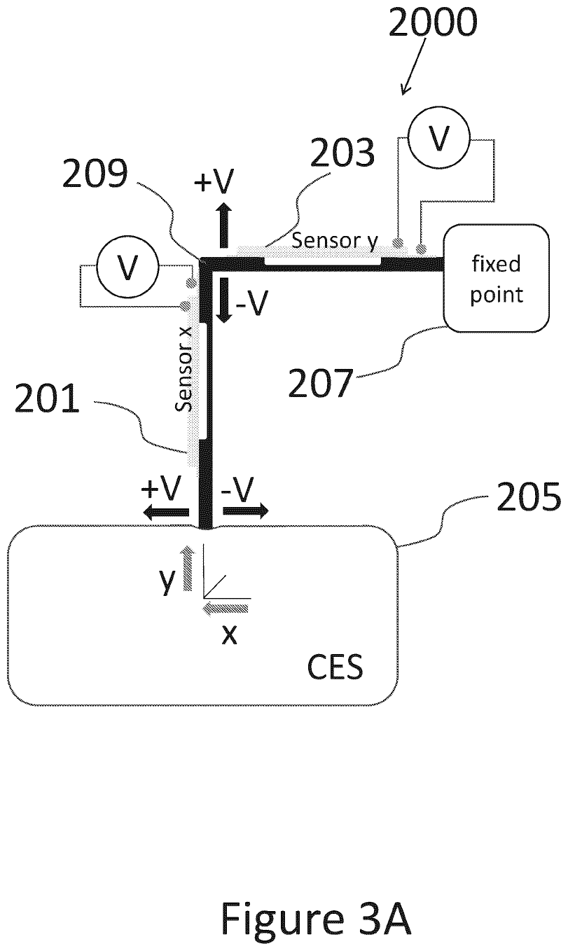

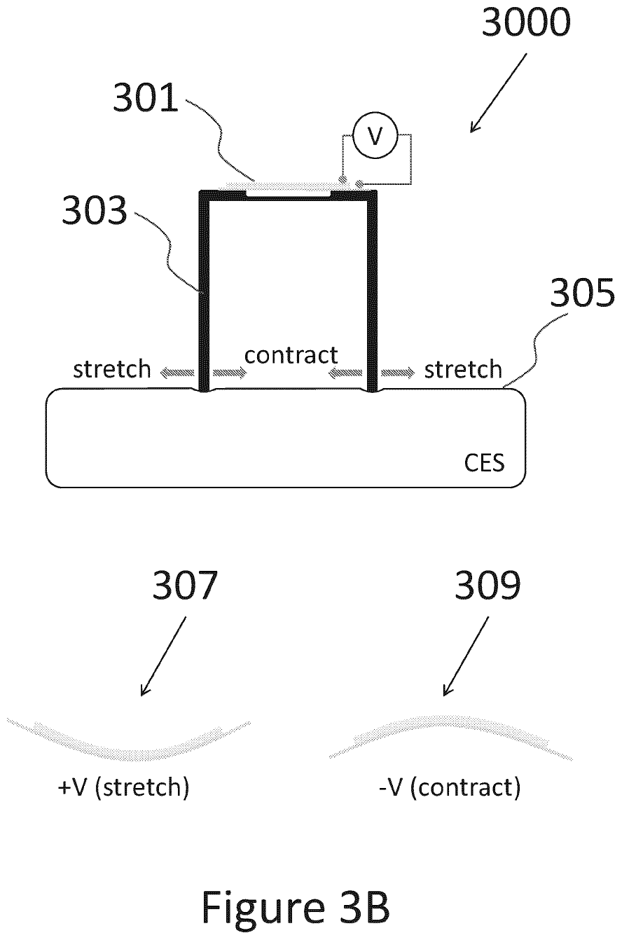

FIG. 2 shows how sensors can be arranged 2000 using two piezoelectric sensor elements 201 and 203 housed in a firm but flexible scaffolding 209 that connects a CES 205 to a fixed point 207, to respond to movements of the CES surface in left/right plane (x-direction movement of CES 205 captured by sensor x 201), and up/down of the CES surface plane (y-direction movement of CES 205 captured by sensor y 203).

FIG. 2B shows how one sensor 301 can be configured in an arrangement 3000 in a firm but flexible scaffolding 303 that responds to stretch and contraction between two points on the body's surface 305. This is achieved by the direction of bend, which results in either positive 307 or negative 309 voltage that is evoked by the distorting piezoelectric crystal in response to stretch or contraction.

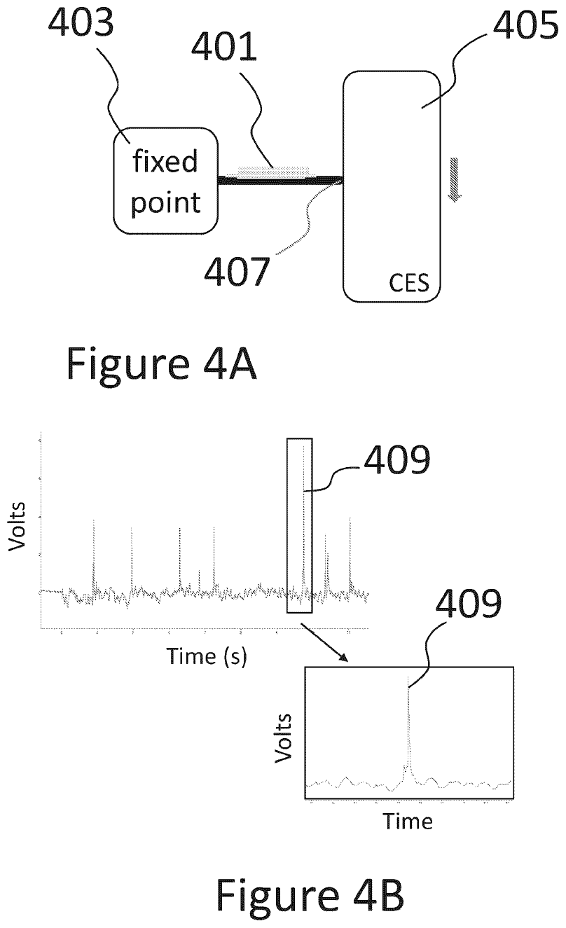

FIG. 4A shows how one sensor 401 can be arranged in a firm but flexible scaffolding that is anchored to a fixed point 403 relative to a CES 405. The tip of sensor scaffold 407 is in contact and orientated perpendicular with the CES 405. When the CES is dragged across the tip of the sensor scaffold in one direction, there are small voltage changes as the scaffold induces small bends of the piezoelectric sensor element. However, depending on the flexibility of the scaffold and the contact properties between the scaffold and the CES, the bending of the scaffold reaches a point where it can no longer be sustained, where it breaks contact and snaps back to its original perpendicular position. This microslip event results in a large, higher-frequency voltage spike 409 as illustrated in FIG. 4B.

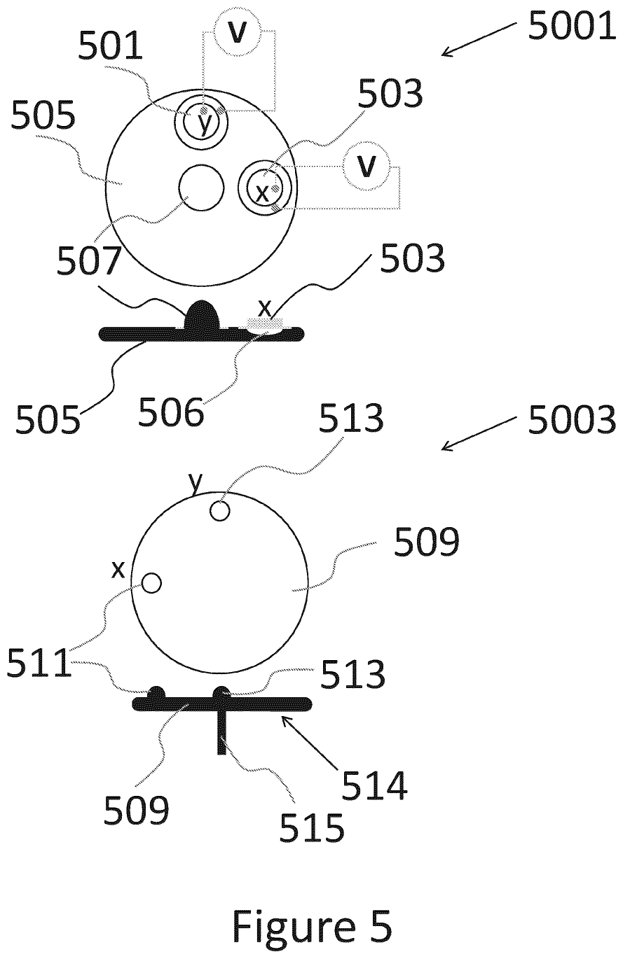

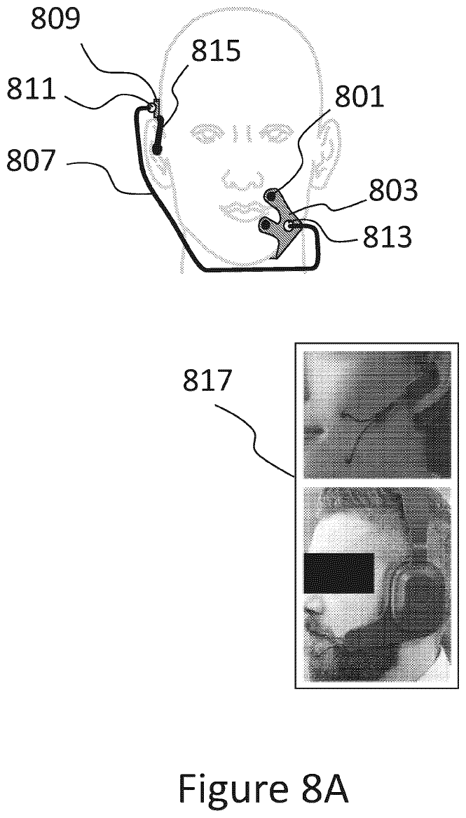

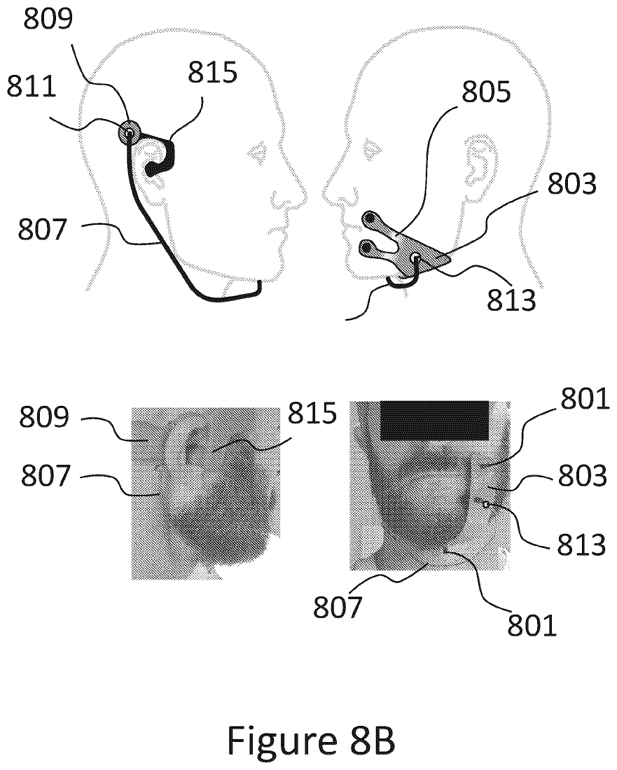

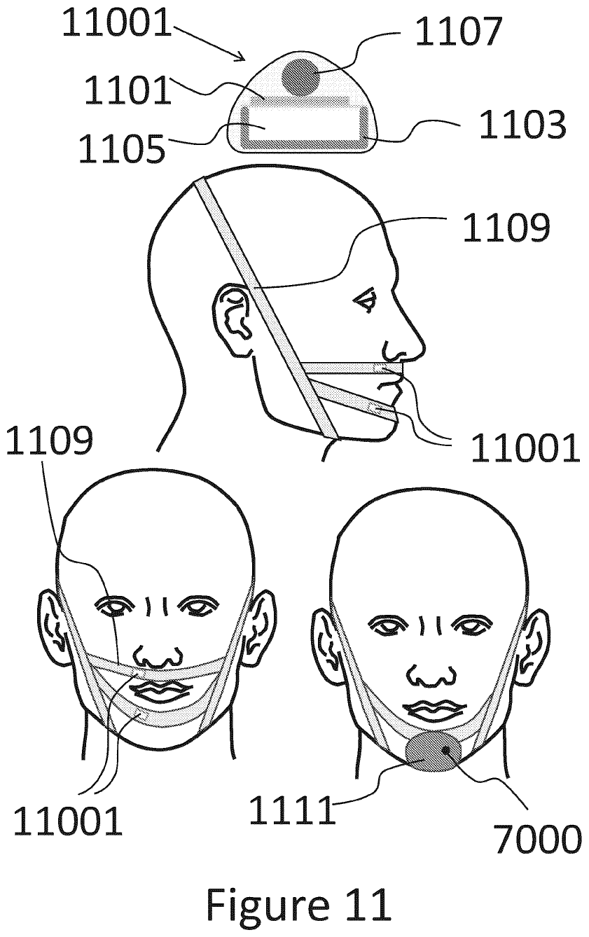

A user interface for sensing human communication expressions is shown in FIGS. 8A and 8B, and in FIG. 11. The user interface has at least one sensor module configured to sense communication expressions of a user and output a sensor data signal (described with reference to FIGS. 5 to 7). The user interface also has a support structure adapted to be worn by a user, such as a head band, harness, adhesive, etc. The support structure is configured to hold the at least one sensor module relative to the user's body so that the at least one sensor module senses the communication expressions of the user.

The at least one sensor module may comprise: a first sensor component having one or more sensor elements; and a second sensor component having one or more reciprocating components for activating the one or more sensor elements; and the first sensor component and the second sensor component may be positioned relative to one another in the sensor module so that communicating movements of the user cause the reciprocating components to move relative to their respective sensor elements thereby causing the sensor elements to sense the communicating movements of the user. FIG. 5 shows how two piezoelectric sensor elements 501 and 503 can be arranged into two rigid non-flexible disks 505 and 509 (facing surface and side views shown for each disk) to provide three axes of movement. This example embodiments shows the sensor components as disks, but it will be understood that other suitable shapes and configurations may be used such as a rectangular prism etc.

It will be appreciated that the sensor component may comprise one or more sensor elements, with more sensor elements increasing accuracy (due to more data being collected from the user's movements), but also increasing computational complexity and the associated processing lag and cost. The inventor has found that having between 2 and 10 sensor elements per sensor component works well.

The figure shows two halves of the assembly for a single sensor housing. In this example, the first sensor component in the form of disk 505 houses two piezoelectric sensor elements relative to a resilient member. In this example, the sensor elements are centred over a recess 506 that permits distortion of the piezoelectric crystal when it is depressed into the disk. This recess 506 can be filled with a gas such as air to change the flexibility of the sensor element.

The sensor component(s) may comprise a support configured to allow, control, limit, and/or otherwise define movement of the sensor component. The first disk 505 includes a support or interface component in the form of a central protrusion 507 configured to allow the disk to freely rock in any direction when an abutting surface opposes the protrusion. The interface component is configured to guide a relative position and relative movement between the first sensor component and the second sensor component. When a user's communication expressions affect the sensor module (for example when movement is detected), the interface component causes relative movement between the first sensor component and the second sensor component in such a way that the one or more reciprocating components activate the one or more sensor elements.

In some embodiments the sensor module may comprise a housing, and the interface component may alternatively or additionally move against the housing when the user performs a communication expression, causing the sensor elements to distort.

The second sensor component includes one or more reciprocating members configured to operatively abut and move relative to the first sensor component. In the exemplary embodiment, the second disk 509 houses two small protrusions 511 and 513 that are designed to interface with the two piezoelectric sensor elements when the two disks are abutted.

The second sensor component is configured to retrieve motion information from the user. For example, the second sensor component may include a user interface surface 514 with one or more structures adapted for placement adjacent to the user's body and/or adapted to retrieve information from the user. For example, the user interface surface 514 of the second sensor component may include one or more sensing structures, for example in the form of one or more resilient, flexible, movable, fixed and/or rigid cantilevers, filaments, ridges, needles, walls, bumps, etc. In the exemplary embodiment, on the reverse side of the second disk is a protruding arm 515 for contact with the CES 603.

The second sensor component may comprise a cantilever that abuts the user and transfers the user's movement to the at least one sensor module by causing the second sensor component to move relative to the first sensor component when the user moves, thereby enhancing a directional sensitivity of the at least one sensor module.

Using 2 piezo crystals is only exemplary, for example increasing the number of sensor elements around the disk would increase the direction selectivity precision of the sensor unit. One or more sensors, with different shaped opposing protrusions (e.g. with asymmetrical profiles) could be arranged around the sensor component.

The resilient member may be configurable. For example, changing the pressures inside the recess 506 provides tuneable options that can generate more information as required about the movement, such as introducing an intended bias to direction selectivity and/or sensitivity.

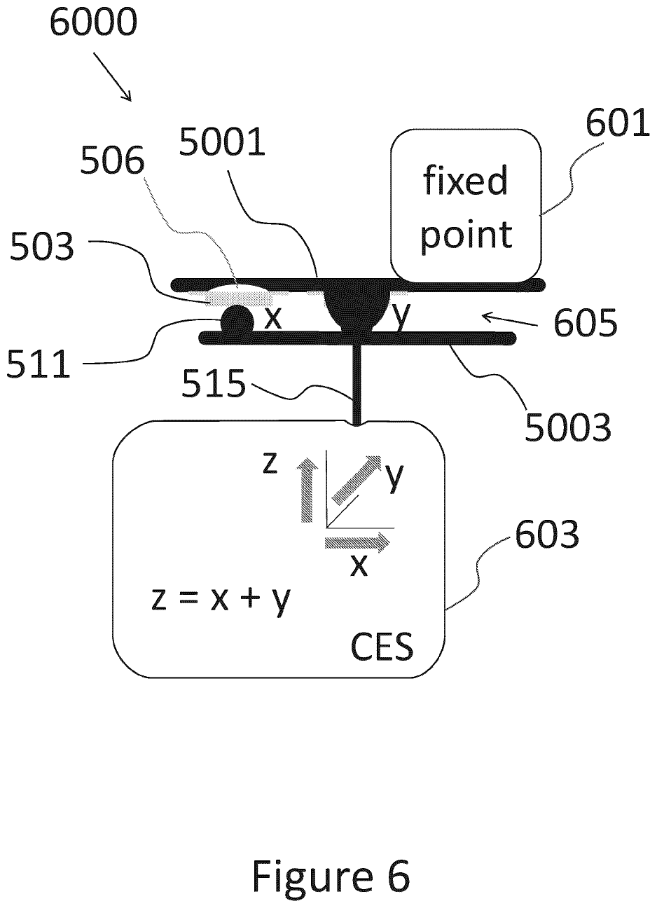

FIG. 6 shows a sensor module 6000 assembly that includes two sensor components 5001 and 5003. In this embodiment the sensor components are in the form of disks. In other embodiments, the sensor module may comprise more than two sensor components, and may comprise an even or odd number of sensor components. The sensor components are positioned relative to one another in such a way that the sensor elements and their respective reciprocating members are operatively positioned relative to one another to transfer movement-related indicators from the user's body to the sensor elements on the first sensor component via the reciprocating members on the second sensor component.

The exemplary embodiment of a sensor module is adapted so that three dimensions of CES movement information can be monitored, tracked, and/or sensed. Sensor components 5001 and 5003 are positioned relative to one another so that the sensor elements and their respective reciprocating members are operatively paired. In the exemplary embodiment, the piezoelectric sensor elements are opposing their respective protrusions on the opposite disk (503 with 511 and 501 with 513).

The sensor components are resiliently held together, for example an internal region 605 between the first and second sensor components may include a resilient, flexible, compressible and/or otherwise non-rigid interfacing. For example, the internal region may be filled with a flexible adhesive (for example silicone rubber). Sensor component 5001 is attached to a reference point being a fixed point 601, and the protruding arm 515 (or in its absence, the disk 5003) is placed adjacent, onto, or otherwise relative to a part of the user's body (e.g. CES) that may move relative to the fixed point. Left/right movement (x-direction) of the CES surface 603 results in the first piezo crystal 503 being depressed into its opposing recess 506, or being lifted out of the recess because of the adhesive material, thereby resulting in +V or −V respectively in the sensor element 503. Backward/forward movement (y-direction) of the CES surface 603 results in the second piezo crystal 501 being depressed into or lifted from its opposing recess, thereby resulting in +V or −V respectively in sensor 501. Thus, sensor element 503 responds preferentially to left/right (x-direction) movement, and sensor element 501 responds preferentially to backward/forward (y-direction) movement. In the event of up/down (z-direction) movement of the CES surface 603, both sensor elements 503 and 501 respond together by being depressed into their respective opposing recesses, or recoiling back, both producing voltage changes. Thus, in this embodiment, information about CES up/down movement arises from a combination of signals from two sensors 501 and 503, whereas backward/forward and left/right movements are signalled primarily by one sensor.

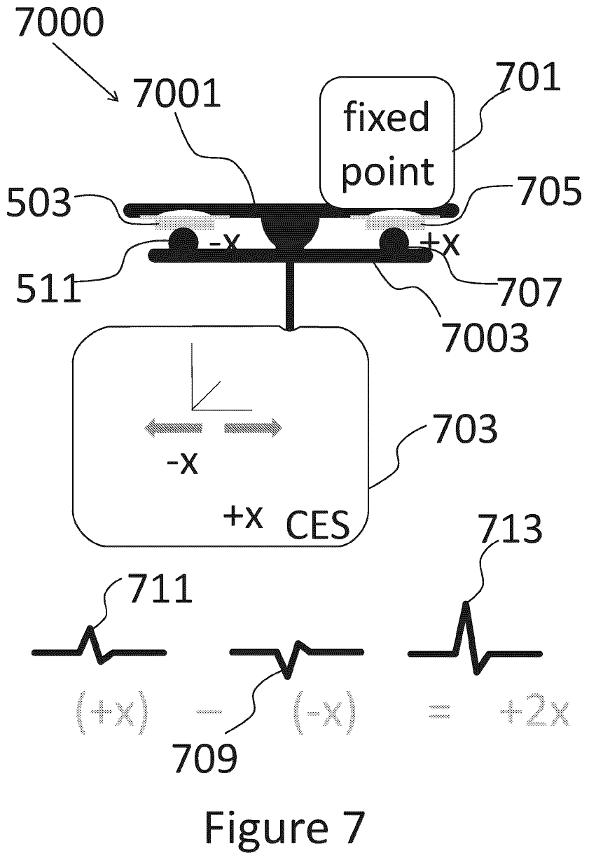

FIG. 7 shows an assembly 7000, similar to assembly 6000, but where there is an additional, third, piezoelectric sensor element 705 positioned on the opposite side of the sensor housing disks 7001 and 7003 to the first piezoelectric sensor element 503. Both the first and third piezoelectric sensor elements 503 and 705 are orientated to respond to the movement of the CES 703 in the x-direction, such that when the CES 703 moves to the right, the third piezoelectric sensor element 705 is pressed by the opposing protrusion 707, while the first sensor element 503 has its protrusion 511 moving away from it and flexed in the opposite orientation.

The effect of movement of the CES 703 in the x-direction to the right is that the first piezoelectric sensor element 503 (illustrated on the left side in FIG. 7) generates a first signal 709 that is inverted compared to the third signal 711 generated by the third piezoelectric sensor element 705 (illustrated on the right side). Therefore, to increase the sensitivity of the signal for x-direction movement, the first signal 709 generated by the first piezoelectric sensor element 503 can be subtracted from the third signal generated by the third piezoelectric sensor element 705, resulting in a difference signal 713 that is two times the amplitude compared to the third signal 711 from a single piezoelectric sensor element for representing movement in the x-direction.

The sensor module may thus comprise a pair of sensor modules positioned relative to one another and sensor signals from the pair of sensor modules are combined to amplify the sensor data signal.

FIG. 8A and FIG. 8B show how the sensors can be held in place onto the CES of the face. The sensors 801 may be housed in a sensor support structure 803, 817 that may be rigid, or flexible, or a combination of both, or have a gradient of flexibility and rigidity 805, 817. A sensor support structure 803, 817 may hold one or more sensors over various locations on the head and neck, and may be shaped to go around different surfaces of the head, such as under the jaw and extend up and around the mouth, which would permit the sensors responding to the movement of the tongue, lips and chin 803. The sensor support structure may also support sensors around the eyes, cheek, forehead and other structures that might respond to a variety of facial expressions, such as smiles, frowns, sadness, anger and the like. The sensor support structure 803 is held in place using a retention component 807 that is connected to an anchor component 809. The retention component may have specialised connecting points between the anchor component 811 and the sensor support structure 813 which may ensure stable anchoring and appropriate distribution of forces across the sensors in contact with the CES. Thus, the anchor component 811 and the sensor support structure 813 may have a single point of contact on their respective articulation joints, such as a ball joint, or it may have multiple points that restrict the degrees of freedom of the articulations, such as a hinge joint, or a fixed joint. The anchor component may be connected to a hearable device (such as a small speaker) to deliver sound to the ear canal 815, 817 of the user, or the anchor point may be incorporate a hearable device itself, such that the anchor point is located in, or around the entrance of the ear canal 817.

The sensor devices described herein may include a user interface configured for input and/or output from and/or to the user, or a recipient. In some embodiments the user interface includes an audio output interface, for example a speaker. In some embodiments the user interface includes an audio input interface, for example a microphone.

The retention component may hold the sensor support structure in place by tension from pressing on opposing sides of the head, for example retention component 807, or the support structure itself may also contain the anchor point such as examples shown in 817.

In the case of a retention component design such as retention component 807, it may be collapsable, extendable, and/or auto-assembling. Examples of these designs may include telescopic extensions of inter-connected tubular structures. The tubular structures could be open on one side or closed, and they could contain cords inside that are under tension. Such cords could facilitate self-assembly, such as an elasticated cord so that when the telescopic sections are pulled apart, the elastic cord pulls them into their assembled structure. In some embodiments, at a point along the retention component 807, sensor support structure 803 and/or anchor component 809, there may be a mechanism, such as a ratchet, to wind the cord, so that the amount of tension in the retention component can be controlled by tightening/loosening the cord. This also enables adjustment and fitting of the sensor support structure for different head shapes and sizes so that the contact on the face can be adjusted accordingly. The cord may also contain electrically conductive components such that the hearable component at one end of the retention component can be in electrical contact with the support structure at the other end of the retention component.

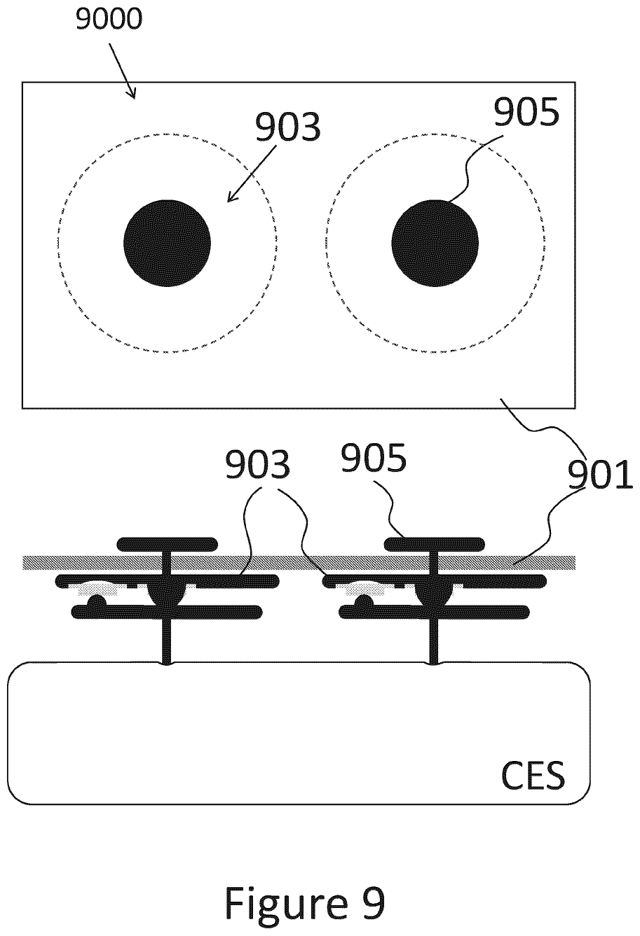

FIG. 9 shows a sensor device 9000 having a support structure in the form of a piece of fabric 901 with elastic properties, illustrated from top and side views, with two 3-axis sensors 903, configured similar to assembly 6000, but that are anchored inside the weave of the fabric, rather than fixed to a solid object as shown in FIG. 6. An attaching means, in this example in the form of a disk 905 with a connecting arm secures the sensors 903 inside the fabric, similar to a button inside a buttonhole, secures the sensors to the fabric support 901. This configuration enables the sensors to provide information about the relative positions of the two sensors to each other, and the relative movement of the underlying CES, similar to arrangement 3000.

The user interface may thus comprise two sensor modules held relative to one another via a flexible and/or elastic fabric, and the two sensor modules are configured to sense relative positions of the two sensor modules relative to one another, the relative positions being indicative of the user's movement.

This sensor arrangement will also respond to distortions of the fabric from regions away from the sensor. Changing the elastic properties of the fabric, or applying non-uniform elasticity inside the fabric, can change the behaviours of the signals in response to fabric stretch.

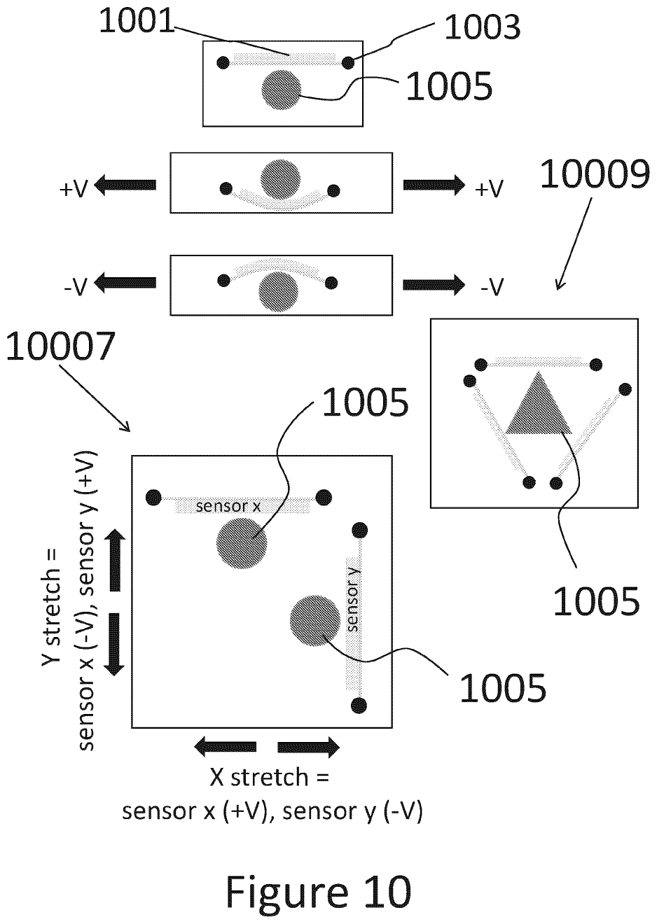

FIG. 10 shows other embodiments of the use of piezoelectric sensor elements 1001 anchored or attached onto various pieces of fabric with elastic properties. In these arrangements, the piezoelectric sensor elements are anchored at anchor points 1003 to the fabric at strategic points (selected based on the appropriate muscles, skin surfaces, etc., for the relevant communication mechanism being monitored). The support structure can thus be configured to hold the sensor modules relative to one or more specific speech articulators of the user.

Furthermore, the elastic fabric contains firmer regions 1005 of varying elasticity and of various shapes and locations relative to the piezoelectric sensor element location and orientation, configured for the purpose of monitoring the user's movements and sensing CES movements that convey a communication by the user. The direction of tension in the fabric can therefore cause a piezo crystal to generate a positive or negative voltage across its surface, depending on the relationship of the stretch of the elastic fabric, its non-elastic components 1005, and the positioning of the piezoelectric sensor element. The fabric can have various regions of altered elasticity as well as sensor element configurations (10007, 10009) to generate different signal behaviours in response to movement of the underlying CES near or away from the sensors. These different behaviours may be induced by introducing something physical into the fabric weave, such as solid plastics (e.g. 1005) and/or adhesives that immobilize and/or or retard the fibres' movement as the fabric undergoes stretch and/or contraction.

FIG. 11 shows how piezoelectric signals can be obtained using elasticated straps for locating sensors on CES. One example of a suitable sensor for this application may include a piezoelectric sensor element 1101 held over a small box 1103 with a resilient member, for example comprising an air space 1105, and a pusher 1107, for example in the form of a ball, cube, or other solid and relatively incompressible mass, positioned relative, adjacent and/or abutting the piezoelectric sensor element 1101. The pressure inside the air space 1105 may be adjusted to modify the sensor response. This package 11001 is then embedded into a silicone material, which is subsequently attached onto the straps 1109 that overlay a CES. The straps may have elastic or inelastic components. Another embodiment may incorporate a more rigid support structure, such as a chin cup 1111, that is held in place with elasticated straps. This may permit other sensor types, such as 7000, which may hold an array of sensors above the chin and under the jaw as examples. Movement by the user will cause the mass 1107 to activate the sensor element 1101 because the sensor module package 11001 is held in place against the user's body via one or more straps 1109.

Another approach to capture intended communication information associated with an intended communication from the body is the use of optical sensors. In prior art methods cameras are used to capture 2D images in a time series for reading speech from under the chin. In contrast, the novel approach described herein captures 3-dimensional information rather than decoding information from 2-dimensional images. The method described herein uses the distance information determined between some reference point and the CES. As the CES change shape over time, the distance changes of individual points across the CES can be used to infer elements of intended communication expression.

One optical approach to determine distance uses Time-of-Flight (ToF) information. This approach emits light at or near a light detector (i.e., positioned relative to an associated light detector), and the time taken for the light to travel from the point of emission to hitting the CES and bouncing back to the light detector is measured and used to calculate the distance.

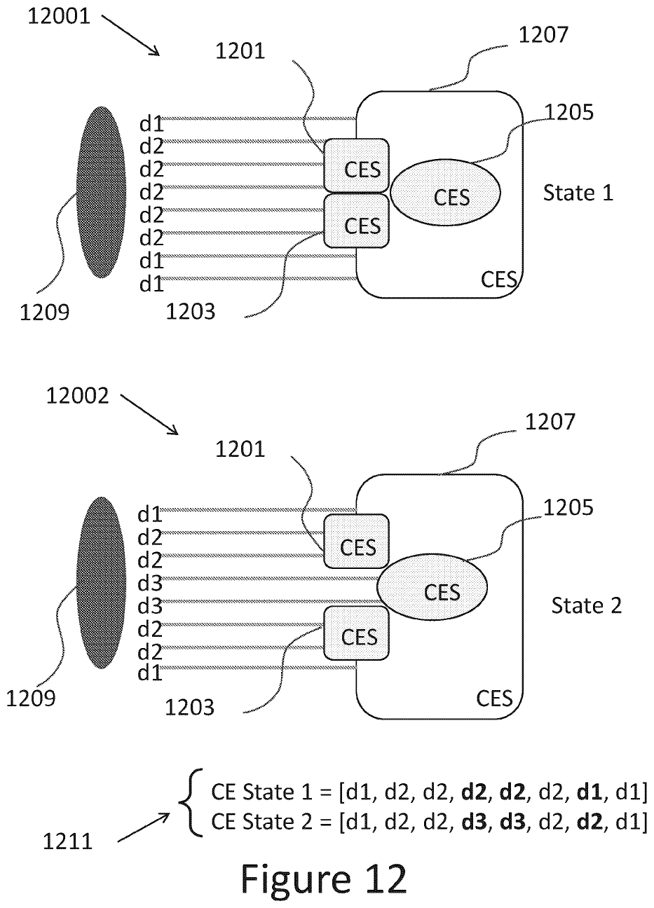

FIG. 12 shows a first state 12001 and a second state 12002 of multiple CES in two distinct arrangements. In the exemplary first state 12001 the top 1201 and bottom 1203 lips are pressed together, such that the tongue 1205 is hidden from the outside world, as it remains inside the oral cavity 1207. The distance, measured by ToF from the various points on the body to an optical capturing unit 1209 will vary, and a vector of these distances can be used to represent this state. In a second state during communication expression 12002 there is a longer optical pathway for a beam of light to travel to the tongue 1205 when the lips 1201 and 1203 are parted, which is otherwise occluded during the first state 12001. These two communication expression states 12001 and 12002 therefore can each be described as a vector representing the light pathway distances across the surface of the subject 1211. Patterns from these vectors can be learned by a machine learning algorithm to determine the state, and subsequently, a sequence of these states can be used to extract intended communication information.

In another embodiment of an optical approach, distance measures are determined with the use of binocular images, where a time series is made for two images that are captured simultaneously using a stereo camera that captures the images from two slightly different horizontal positions. A disparity map is created which represents the difference in the positions of corresponding features in the left and right images. Depth can then be determined using the disparity values to calculate the depth of each point in the scene. The depth Z can be calculated using the formula:

$$Z = ( f * B ) / d$$

where f is the focal length of the camera, B is the baseline distance between the two cameras, and d is the disparity i.e. the difference in position of corresponding points in the left and right images. The larger the disparity, the closer the object. These data can then be used to generate a vector of distances representing points on the image, similar to that as described for the ToF 1211, and as such, that the sequence of states can be used to extract the intended communication information, which can subsequently be passed to a machine learning algorithm to associate the vectors to components of the intended communication.

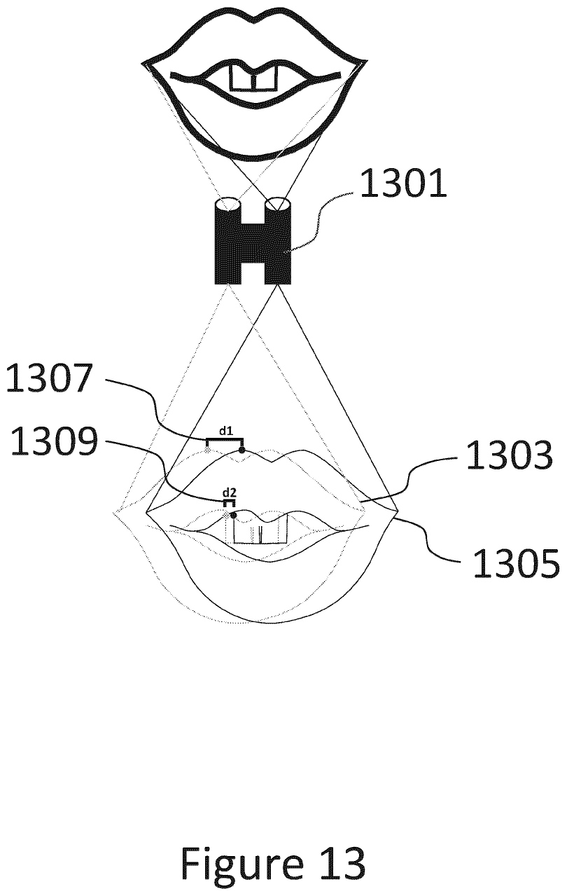

FIG. 13 shows an optical approach to determine depth information using a stereo binocular camera 1301 that generates two similar, but slightly displaced images 1303, 1305. The same point on the body is represented at slightly displaced locations within each camera's field of view, which depends on its distance to the stereo camera. An object that is closer to the stereo camera, such as a point on the lips 1307, will have a greater binocular disparity than an object, such as the teeth 1309, which is further away inside the oral cavity.

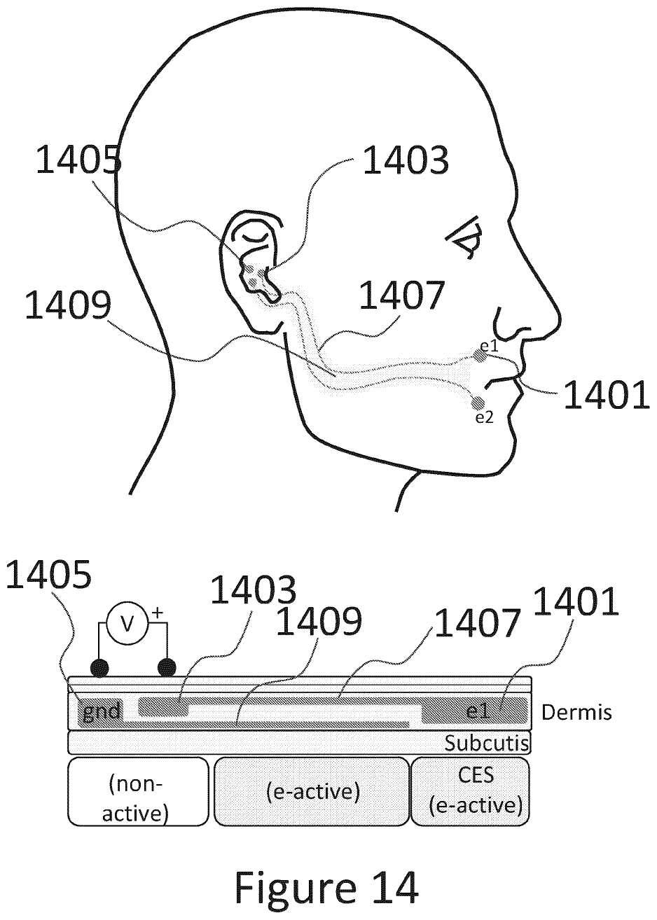

FIG. 14 shows another way to extract signals from CES using a more permanent approach. Here subcutaneous electrode elements 1401, 1403, 1405 and subcutaneous conductive pathways 1407, 1409 are implanted under the skin. The active electrode 1401 is implanted over a CES, such as a nerve, muscle or other elements of an excitable cell or tissue. When the CES is active, an electrical potential is carried from the active electrode 1401 along the conductive part 1407 to a subcutaneous pickup electrode 1403, which lies under the skin over a non-active part of the body, such as the cartilage of the pinna. Another conductive pathway acts as an electrical shield 1409 that is connected to a different electrode 1405. An external device, such as an amplifier, analogue digital convertor, or both, can therefore sit over the electrode pairs 1403, 1405, where a potential difference can be captured to provide a signal representing the CES activity that lies under the active electrode 1401.

In another embodiment, the amplifier, analogue digital convertor, or both, is embedded under the skin, which transmits the amplified and/or digitised signals to an external processor. In another embodiment, the processor is also embedded under the skin.

Each active electrode and its corresponding conduction pathway and pickup electrode are electrically insulated from the rest of the system. These conductive elements could include metal ink that are applied by a tattooing procedure, or they could be insulated wires with naked ends at the electrode and pickup sites. They may take a serpentine pattern 1407 though the body which is designed to provide flexibility and strain relief for accommodating movement and thermal expansion without causing undue stress or breakage. The advantage of this approach is that it reduces the complexity of reading electrical signals from skin electrodes (wet or dry) that are placed across different parts of the body which may result in skin impedance changes of differing amounts over time and with different humidity and temperature or sweating conditions.

Another approach is to implant mechanical sensors under the skin, such as a piezoelectric element configured for subcutaneous application. This approach is similar to the EMG approach shown in FIG. 14, but a mechanical-derived signal is captured from under the skin rather than an electrical signal. As the mechanical-derived signal is converted into an electrical signal, it can then be treated in the same way as an EMG signal.

A PSLI device, that is made up of one or more of these sensors and/or sensor types, can be constructed.

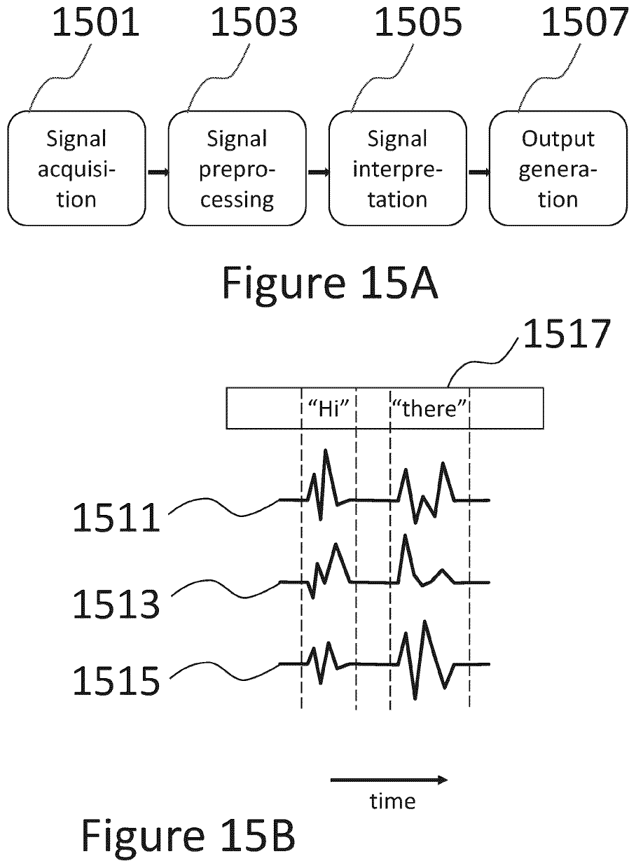

FIG. 15A shows one embodiment of a PSLI device that includes four functional modules:
a. A signal acquisition module 1501,
b. A signal pre-processing module 1503,
c. A signal interpretation module 1505, and
d. An output generation module 1507.

Signal acquisition is a process for converting any of the described signals into some useful output for communicating the user's intended communications. Signal acquisition at 1501 may capture signals, such as shown in FIG. 15B, from one or more of the sensors described (e.g., housed in a disk arrangement, fabric or rigid scaffolding etc.), which may belong to one or more of the sensor types described (e.g., mechanical, electrical, and/or optical). The signal acquisition module 1501 therefore contains the necessary components to capture mechanical, electrical, and/or optical signals, or a combination of these. For mechanical sensing, the module may be configured to capture surface distortions related to speech articulation and/or hand/arm gesturing. These could be movements on the skin, fabric, or other solid components in proximity to, or in contact with, the articulatory organs of speech or the forearm as examples. In some embodiments they could be implanted under the skin and/or around muscles. As an example, this may be achieved through piezoelectric sensors, which convert changes in mechanical features of the CES into electrical signals. This helps the system capture mechanical information from surfaces. For optical sensing, a 3D representation of the moving surfaces can be captured. For electrical sensing, electrical potentials may be captured at the pickup electrodes. The system could collect one or more of these signals from one or more sensing approaches.

The captured signals may undergo an optional signal processing step at module 1503, and depending on the signal type, the signal processing step may be absent or more involved. Typically, mechanical sensing from the piezoelectric sensors do not require much, if any, signal processing, however, optically and electrical derived signals may require some signal processing steps, for example noise removal and normalisation, that are standard in the field for those signal types.

The next step is signal interpretation at module 1505. In some embodiments this module includes a machine learning submodule configured to execute a machine learning algorithm trained to recognise unique signature patterns generated by the one or more sensing modalities during the different thought expression states. The algorithm interprets the signals and determines part or all of the user's intended communication thought, as shown in FIG. 15B.

The recognised patterns are translated into some useful output by output module 1507 in order to communicate the intended communication for the relevant application. The generated output may be a part of, or a complete thought expression. Typically, a thought expression may be represented by a word, a sentence, phrase, command/instruction, an emotional state, and presented as text, an emoji, synthesized speech, or control signals for a computer, app, or device such as computer code. These expressed thoughts could then be used for communicating ideas to others or for controlling computers, apps or devices via some digital technology, for example another device or the internet.

In some embodiments, the digital interfacing of the devices described herein include artificial intelligence (AI) interfacing. One example is for humans to communicate to an AI system, using natural language. The AI interface may harness a trained language model, to separate the intended communications into those intended as communication for another human, or for communication to some digital technology, such as a computer, app, or device.

FIG. 15B shows and example of signals from 3 sensors 1511, 1513 and 1515 that are captured during an intended communication 1517. The sensors may be placed on one or more CES. If there are more than one signal being acquired, the acquisition of all the signals is synchronised, such that all captured signals represent the same time window. The intended communication 1517 can therefore be represented by one or more of the sensor signals shown. Each signal may represent a unique signature for part or whole of the intended communication 1517. The signals may arise from mechanical-based sensors, such as the piezoelectric sensors described above, or similar sensors like strain gauge sensor signals, or the signals may represent distance information such as those described for the ToF and binocular approaches, or the signals may be derived from electrical signals, such as the EMG approach described above, or the signals may be a mix of one or more mechanical, optical, and/or electrical sensor signals.

These unique signatures are used to train a machine learning algorithm, such as a neural network, so that the machine learning algorithm learns to associate the one or more unique signatures to a component of the intended communication expression. Once trained, the machine learning algorithm will classify signals from the same one or more sensors into the components of the intended communication expression when they are presented to the classifier.

The signals from the classifier may then undergo another processing step whereby the raw inference from the machine learning algorithm is passed though another algorithm that interprets the classified signals and prepares them for their desired output. For example, if the intended communication is destined for a text message application, the processor formats the machine learning inference to the appropriate text format. In another example, the intended message may be a voice message, in which case the processor formats the machine learning inference into a synthetic voice to be outputted appropriately. In another example the intended message may be a command to interface with another device, so the processor will format the machine learning inference into the appropriate code that the device can understand.

The intended communication may also include a combination of communication intended for another human as well as a device or software application. The processor therefore may be required to separate the intended communication inference for different final destinations or categories.

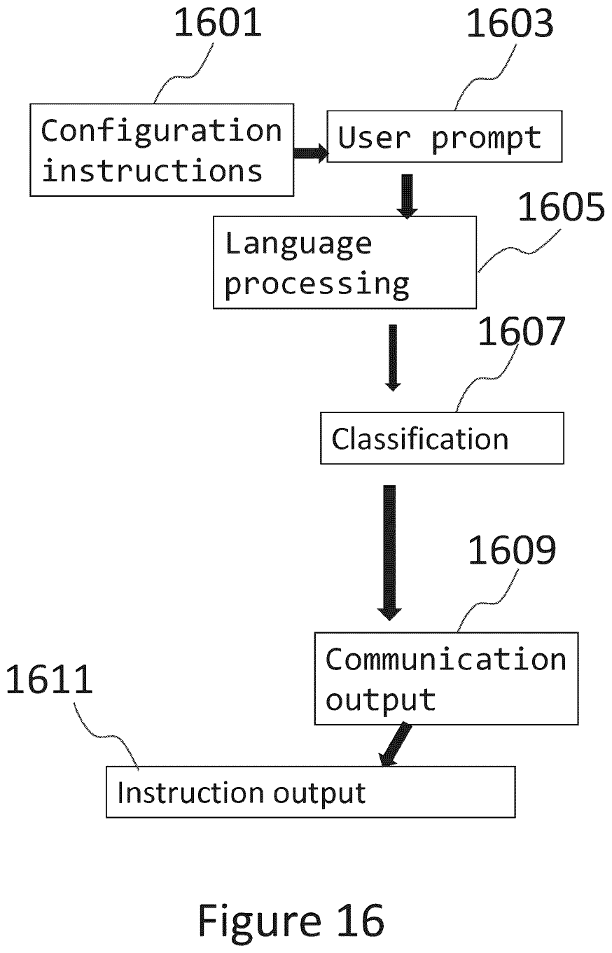

FIG. 16 shows a flow diagram of an example describing a high-level overview of an AI system where a large language model (LLM) processes user prompts to separate sentences into two categories:
conversational content intended for another human, and
instructions for an app (such as a smartphone app) or device control, in this example for controlling a drone.

The method begins by providing configuration instructions to the LLM 1601 (see Table 1 for details). This message instructs the LLM to classify and format messages into two distinct categories: conversational messages and app or device control commands. Once the language model is configured, a user provides a prompt at 1603 (see Table 2 for details) which was extracted from the preceding signal interpretation step 1505. This prompt contains a message which could contain conversational content intended for a human recipient and/or instructions for controlling an app or device. The language model at 1605 processes the user's prompt (see Table 3 for details). By using its training and the guidance provided by the configuration instructions, it interprets the intent of each sentence in the prompt. The language model then classifies at 1607 (see Table 4) each sentence based on its intent and formats it appropriately. Conversational content may be prepared in a text format, which may later be used for speech synthesis, while device control commands may be formatted into a specific coding language. The classified and formatted outputs are delivered to their destinations at 1609, 1611. Conversational messages are sent to the relevant communication channel at 1609 (see Table 5 for details), and app or device control commands are sent to the specific app or device to be controlled at 1611 (see Table 6 for details). This flow diagram encapsulates the process of using an LLM to handle diverse types of content, from human conversation to app or device control, highlighting the potential of LLMs in interacting with both humans and machines in their own “languages”.

TABLE 1
AI_instructions =
″″″
You are an AI assistant whose role is to identify what is part of a normal conversation
verses what is commands to fly a drone. For instructions that are for drone control, you
are to format the response using the following, and insert the number, formatted in cm
for a distance or degrees if an angle, into the parentheses. If a flip is called, the insert
a letter instead of a number into the parentheses. The commands are as follows:
‘‘‘

| | |
|---|---|
| drone.takeoff( ) | # makes drone take off |
| drone.land( ) | # makes drone land |
| drone.move_forward(x) | # makes drone move forward by x cm |
| drone.move_back(x) | # makes drone move backwards by x cm |
| drone.move_left(x) | # makes drone move left by x cm |
| drone.move_right(x) | # makes drone move right by x cm |
| drone.move_up(x) | # makes drone move upwards by x cm |
| drone.move_down(x) | # makes drone move down by x cm |
| drone.flip(″f″) | # makes drone do a forward flip |
| drone.flip(″b″) | # makes drone do a backwards flip |
| drone.flip(″l″) | # makes drone do a flip to the left |
| drone.flip(″r″) | # makes drone do a flip to the right |
| drone.rotate_clockwise(x) | # makes rotate in the clockwise direction by x degrees |

drone.rotate_counter_clockwise(x) # makes rotate anticlockwise by x degrees
‘‘‘
For parts of the prompt that are not directed towards controlling the drone, you are to
separate those into another format and fix any grammatical or spelling errors. Your
answer should separate the parts that are for the drone and parts that are part of the
normal conversation into a specified format.
For example, if the prompt is as follows:
‘‘‘
Hi there, Im going to demonstrate how I can have a normal conversation and also give
drone instructions, while not getting them confused. Drone go forward by 1 meter. Now
it should go forward by one meter. ok, drone now go back by 20 cm. Now it should go
back by 20 cm. So that's it, isn't it great! Drone, do a flip to the right. Now rotate to the
right by 90 degrees.
‘‘‘
That prompt should give the following output:
‘‘‘
chat Hi there, I'm going to demonstrate how I can have a normal conversation and also
give drone instructions, while not getting them confused.
comm drone.move_forward(100)
chat Now it should go forward by one meter.
comm drone.move_back(20)
chat Now it should go back by 20 cm. So that's it, isn't it great?
comm drone.flip(″r″)
comm drone.rotate_clockwise(90)
‘‘‘
″″″

TABLE 2
prompt = “hi there so I'm going to show off my new toy. Drone take off
move forward by 1 meter turn to the left by 45 degrees do a flip
backwards and then land. So what do you think pretty cool hey″

TABLE 3

AI_output = intention_ discriminator( prompt, AI_instructions,
trained_LLM )

TABLE 4

```
7-element Vector{SubString{String}}:
  “chat Hi there, so I'm going to show off my new toy. ”
  “comm drone.takeoff( )”
  “comm drone.move_forward(100)”
  “comm drone.rotate_counter_clockwise(45)”
  “comm drone.flip(\“b\”)”
  “comm drone.land( )”
  “chat So what do you think? Pretty cool hey!”
```

TABLE 5

```
for i in AI_output
  response = i[5:end]
  if i[1:4] == “chat”
      synthetic_voice(response)
  else
    evaluate_drone_instruction(response)
  end
end
```

TABLE 6

```
text_to_speech(“Hi there, so I'm going to show off my new toy.”)
  drone.takeoff( )
  drone.move_forward(100)
  drone.rotate_counter_clockwise(45)
  drone.flip(″b″)
  drone.land( )
  text_to_speech( “So what do you think? Pretty cool hey!”)
```

### Applications

The methods described herein, utilizing PSLIs and the novel sensor devices described, have potential applications spanning a wide range of human-digital interfacing scenarios, offering significant advantages over traditional methods such as keyboards, touchscreens, or voice recognition. It enables humans to interface with a wide range of digital technologies that is voice-free, hands-free and eyes-free.

Communication Devices: PSLIs can facilitate silent communication, enabling actions like making calls using silent speech. This application is advantageous in environments with ambient background noise that would typically interfere with voice recognition systems, or where private or covert conversations are necessary. The user may communicate their intended communications to a PSLI system, which could be received by a recipient as synthetic speech.

Text-based Interfaces: PSLIs can be utilized for sending text-based messages, emails, dictation, or any form of digital communication that traditionally requires typing or voice input, enabling an intuitive, eyes and hands-free interface. The key advantage of PSLIs for text-based digital interfacing is that it permits the user to input text into a digital technology at speeds of 3 or more times faster than typing. The advantage over voice-recognition is that it is not encumbered by ambient noise and the content remains private.

Device Control: PSLIs are highly applicable in the field of device control. For example, it can control mobility devices like wheelchairs, drones, or robotic devices without the user broadcasting commands out loud, offering a silent, efficient, and private mode of interaction.

Software Application Control: PSLIs can be used for controlling software applications in a similar way to device control. For example, it could be used to navigate web pages, or smartphone apps in a voice-free, hands-free and eye-free way, which is helpful for improving accessibility of these technologies.

Translation Services: PSLIs can be instrumental in real-time translation scenarios, where a user communicates silently in their native language, and the device generates corresponding text or synthesized speech in another language. This not only facilitates silent communication but also eliminates language barriers. Furthermore, it can be used to translate sign language into text or synthesised speech for recipients who do not otherwise understand sign language. Another example is for changing accents of employees in offshore call centres, so that their accents match the destination of their calls to facilitate being understood in the country they are serving.

Accessibility Technology: For individuals who cannot speak or have difficulties with traditional communication, PSLIs provides a valuable tool. Sign language users can have their gestures transcribed as text or synthesized speech, enabling them to communicate with non-sign language users more effectively. Patients who lost their voice can have their voice restored, blind subjects can interface with assistive technologies without broadcasting their intentions to bystanders as they interface with their devices and computers.

Restrictive communication scenarios: For environments that require breathing apparatus or masks that make verbalising speech difficult or impossible, e.g., snorkelling or protective respiratory apparatus etc, communication expression signals can be extracted from sensors embedded inside fabrics or masks.

Interaction with AI Applications: PSLIs enables silent communication with artificial intelligence applications, such as AI companions. This feature provides a range of potential benefits, from high-level app or device control using natural language to low-level direct translation, as well as private interactions and interfacing with smart devices for general communication. The combination of PSLIs with AI presents many applications that were previously not possible. AI applications can also include personal assistants for a range of applications such as to provide the user with mental health support, technical support, or other assistant roles with the added benefits of the keeping the human-AI interaction silent, private and noise-proof.

These illustrative examples should not be seen as limiting. The PSLI technology, because of its versatility and broad applicability, can find uses in a variety of other sectors and applications where intuitive, silent, and hands-free human-digital interaction is advantageous.

### Definitions

AI: Artificial Intelligence (AI) refers to any system, process, or methodology that enables machines or software to perform tasks that typically require human intelligence. This includes, but is not limited to, capabilities such as learning from data (machine learning), reasoning, problem-solving, perception, understanding natural language, recognizing patterns, and making decisions. AI can be implemented through various techniques, including algorithms, statistical models, neural networks, and rule-based systems, and can be applied to a wide range of applications, such as automation, data analysis, user interaction, and autonomous operations.

AI-interface: An AI-interface refers to any system, mechanism, or method that facilitates interaction between a user (human or machine) and an artificial intelligence system. This includes, but is not limited to, hardware devices, software applications, graphical user interfaces, speech recognition systems, gesture-based controls, and other interactive technologies that enable the input, output, and communication of data and commands to and from an AI system. The AI-interface is designed to interpret user inputs, translate them into actionable data for the AI, and present the AI's responses in an understandable manner, thereby enhancing the usability and accessibility of AI functionalities across various applications and platforms. AI-interface permits humans to use their natural language to communicate thought intentions to other entities, such as other humans, devices, software applications, computers, smart-devices, and the like.

Biopotential: Biopotential refers to the electrical signals generated by the physiological activities of living cells, tissues, or organisms. These electrical signals are produced by the movement of ions across cell membranes and can be measured and recorded from various parts of the body, such as the heart, muscles, brain, and nerves. Biopotentials are typically characterised by their voltage and frequency and are used in various medical and research applications to monitor and analyse physiological functions. Examples of biopotentials include electrocardiograms (ECG) from the heart, electromyograms (EMG) from muscles, electroencephalograms (EEG) from the brain, and electroneurograms (ENG) from nerves.

Biosignal: Any biologically generated signals, which includes mechanical, visual or electrical changes to the body or its structures. The terms biosignals, biological signals and signals may be interchangeable. A biopotential is one example of a biosignal.

Digital technology: Digital technology encompasses any electronic tools, systems, devices, software, artificial intelligence, and methods that utilize digital signals, represented by binary code (comprising 0s and 1s), for the purpose of generating, storing, processing, transmitting, or receiving data. This includes, but is not limited to, computing and smart devices, communication networks, multimedia systems, data storage solutions, software applications, language models, machine learning and artificial intelligence. Digital technology applies to a wide array of fields and industries, enabling functionalities such as automation, connectivity, data manipulation, and interactive user interfaces.

EMG: Electromyography is the electrical biopotential generated when muscles cells are excited.

ENG: Electroneurography is the electrical biopotential generated when nerve or neuron cells are excited.

Intended communication: An element that the user wishes to communicate to the outside world. This may refer to a “thought” associated with an intended communication. This is distinct form an internally generated thought which is not intended to be communicated to the outside world. Intended communications are usually communicated using a language, such as speech or sign language. Intended communications may also include expressions such as facial expressions like smiles, frowns and the like, that may represent an emotional state.

Communication expression (CE): This refers to the expression of thoughts associated with a user's intent to communicate, for example an intended communication, and may include, speech, silent speech, and gesturing anywhere on the body (e.g. face, arms, hands).

Invisible silent-speech: Silent-speech with minimal visible lip movements, for example, like speech from a ventriloquist, but without the vocal component.

Language model: A language model is a type of artificial intelligence model that is trained on volumes of text data. It uses statistical and computational techniques to understand, generate, and manipulate human language to generate human-like text, answer queries, provide summaries, translate languages, analyse sentiment, and perform various other language-related tasks. The term “large language model” typically refers to the size of the model in terms of the number of parameters it has, often in the range of billions or even trillions, which allows it to capture and generate complex language patterns. Language models leverage techniques from natural language processing and machine learning, and they are often built using architectures like recurrent neural networks, transformers, or other deep learning frameworks.

Mimed silent-speech: Silent-speech with lip movements.

Peripheral Silent Language Interface (PSLI): In the context of this disclosure, a PSLI is a more specific SLI that involves the extraction and interpretation of signals from peripheral structures of the body, rather than central structures like the brain or spinal cord. PSLI is grounded in the understanding that intended communications are initially generated in the brain, then propagated through the body as instructions. These instructions are relayed to peripheral organs and are transformed along their path until they reach and influence the external world. PSLI captures these signals on their path to the end organ modifying the outside world, thereby deducing parts or the entirety of the subject's intended communication. PSLI presents a novel means of capturing and translating these intention-bearing signals, providing an innovative method for human-digital interaction that does not necessitate traditional physical interactions. PSLIs can be used to interface with intended speech or sign language.

Sensor: In the context of this disclosure, a sensor is defined as any component or device capable of detecting or measuring a physical property and signalling the results to be recorded and/or interpreted. This term can apply to a single sensing element, which may be a standalone entity, such as a piezoelectric crystal responding to mechanical distortions. It may also refer to an integrated unit or assembly composed of one or more sensing elements, each configured to sense and respond to certain properties, movements, or changes in their environment. The integrated unit or assembly may be housed within or integrated into other structures or devices. These sensing elements or integrated units can be mechanical, optical, electrical, or of any other type suitable for detecting or measuring physical properties. This definition is intended to be inclusive of various types of sensing technologies and is not limited to any particular method or mechanism of detection or measurement.

Silent-speech: Speech without vocalisation, such as whispering or miming speech.

Silent Language Interface (SLI): In the context of this disclosure, SLI is an interface that facilitates the communication of an intention associated with the user's thoughts that they want to communicate, without the use of vocalisation, visualisation, or the use of hands or limbs. SLI involves the employment of interfaces such as brain-computer interfaces and other neurotechnologies that can transduce brain activities into digital signals, which are then transmitted, received, and interpreted by another entity. It will be understood that the methods and systems described herein can be used for audible language, however the term “silent” is used because the focus is not on using a conventional microphone as one of the sensors, although this is of course also possible.

SLIs capture the translation of body elements under neural control, like lip movements or hand gestures. These movements and gestures signify intended communications being expressed somewhere along their efferent journey to the end organ responsible for physical modification of the external world.

Communication-expressing structures (CES): Structures that are altered in a way that is consistent with an intended communication. For example, they may include the articulatory organs, or muscles and/or skin surfaces on the body (e.g. the surface of the face or forearm). A CES often changes its shape or presents with surface distortions as the underlying muscles are recruited from the expression of an intended communication. Lips are an example of a thought expressing structure, because they move as language is being expressed. Electric signals generated by the human body also form part of CES, because electrical signals are generated when a person starts to move and may also be generated even before movement occurs, for example when muscles are tensed in anticipation of potential movement.

### Benefits

The methods described herein use one or more sensor types (mechanical, optical, electrical) on, or in close proximity to, the body, to overcome the complexities of brain-implant SLI approaches as well as EMG sensing instability or surface recordings.

The mechanical sensing captures distortions on surfaces related to communication expression. These sensors may be arranged to respond with directional preference to the orientation of physical distortions on the body and provide unique signatures that represent communication expressions. Capturing mechanical changes on the body for decoding communication expressions from outside the oral cavity as described here have not been described before. The biosignals from the mechanical sensors are of high quality and low noise compared to the classic EMG signals extracted from surface electrodes. Biosignals from the mechanical sensors require less signal processing and lower sampling frequencies, enabling reduced computational requirements for inferring communication expressions. An example of a component of a mechanical sensor is a piezoelectric crystal, and the arrangement of these with respect to their mechanical housing and the CES.

The optical approach described here is novel because to date, no one has detailed the use of 3D reconstruction of images that capture surface changes of CES over time for translating these back into intended communications. 3D information from images enables depth information to be extracted from images to provide additional information about the status of the CES, thereby enabling the translation of communication expressions into intended communications. The use of Time-of-Flight approaches, such as LiDAR, or stereo binocular optics are examples of sensing not seen in existing optical methods of communication expression translation. These sensing approaches enable the extraction of 3D information of CES over time, providing a higher level of nuance and specificity in the communication expressions decoding process.

The electrical approach removes the instability of surface recordings at the active site and their condition pathways by implanting the electrodes under the skin. This reduces the surface recording variance across multiple electrodes as it reduces the recording site to a single location which is more stable because of reduced mechanical and impedance variance. Another approach is to use implantable electrode/amplifier/analogue digital converts under the skin to extract communication expressions information. These approaches are also more stable over time and is designed as a permanent solution for applications such as voice restoration.

Another innovative aspect of the sensors is the external detection of tongue positioning. Whereas prior solutions typically required intraoral sensors to gather this information, the methods described herein provide a more comfortable, non-invasive approach by capturing tongue position information from outside the oral cavity.

Another innovation is the combined use of these sensing modalities in a system which provides a comprehensive solution to communication expressions translation. Its flexibility in the sensor options and placement offers enhanced comfort and convenience to the user. Furthermore, the additional information each sensor type contributes enhances the accuracy that can be achieved by one sensor type alone. The system's ability to improve over time through machine learning algorithms, offers personalized communication expressions decoding, thereby ensuring greater accuracy and ease of use compared to classical approaches.

As an integrated system for communication, the system described herein can be utilised for interfacing with devices using artificial intelligence, which enables a more seamless user interface experience for the user, such that they can control devices and software applications using natural language. The innovation uses artificial intelligence to separate CE-derived signals into those intended for app or device control (e.g. communication to an app or device) and those intended for general communication (e.g. communication to another human).

In summary, the methods described herein provide an improvement in PSLI technology, offering a reliable, less invasive or non-invasive, and user-friendly solution that is adaptable to the unique needs of each user.

### EXEMPLARY EMBODIMENTS

### Example Embodiment 1

Clause 1: A method for interpreting intended communications from a subject, the method comprising:
a. Converting physical changes of thought-expressing structures (CES) into electrical signals using one or more sensors;
b. Processing these electrical signals into data representative of intended communications;
d. Utilizing this data to perform actions or generate output that corresponds to the intended communications.

Clause 2: The method of Clause 1, wherein a sensor includes a piezoelectric or piezoresistive sensor.

Clause 3: The method of Clause 2, wherein the configurations of said sensors are tuneable to extract specific features from the CES.

Clause 4: The method of Clause 1, wherein a sensor includes an optical sensor.

Clause 5: The method of Clause 4, wherein optical sensors provide signals that can be used to extract distance information from a reference point for one or more CES.

Clause 6: The method of Clause 1, wherein the one or more sensors are integrated into a module, housing, scaffolding, or fabric that are in direct or indirect contact with the CES.

Clause 7: The method of Clause 1, wherein the one or more sensors are integrated into a module, housing, scaffolding, or fabric that are in close proximity, but not in contact with the CES.

Clause 8: The method of Clause 1, wherein the one or more sensors include one or more of the following: an electrode for capturing biopotentials, strain gauge sensor, load cells, force-sensitive resistors, force transducer, capacitive sensor, resistive sensor, inductive sensor, magnetoresistive sensor, or acoustic sensors.

Clause 9: The method of Clause 1, wherein the CES are selected from a group consisting of facial muscles, or articulatory organs, or body surface areas, or any combination of these.

Clause 10: The method of Clause 1, wherein the processing of electrical signals involves machine learning algorithms.

Clause 11: The method of Clauses 10, wherein the machine learning algorithms include neural networks.

Clause 12: The method of Clause 1, wherein the processing of electrical signals involves language models.

Clause 13: The method of Clause 1, wherein the processing of electrical signals involves speech synthesis.

Clause 14: The method of Clause 1, further comprising filtering of electrical signals or processed electrical signals to differentiate between intended communication-derived signals and other signals.

Clause 15: The method of Clause 1, wherein the method is used for silent communication.

Clause 16: The method of Clause 1, wherein the method is used for providing accessibility to individuals with disabilities, unique communication needs, restoring voice, or in situations where speech is otherwise obstructed.

Clause 17: The method of Clause 1, wherein the method is used for providing an alternative interface to digital technologies for individuals with visual impairments.

Clause 18: The method of Clause 1, wherein the method is used for communication in noisy environments.

Clause 19: The method of Clause 1, wherein the method is used for language translation and/or for replacing speech that is difficult to understand.

Clause 20: The method of Clause 1, wherein the actions or output generated correspond to inputs traditionally provided through a keyboard, touchscreen, computer mouse, or voice-recognition interfaces.

Clause 21: The method of Clause 1, further comprising using the method to replace a traditional keyboard, touchscreen, computer mouse, or voice-recognition interface between a human and a digital technology.

Clause 22: A method for processing intended communications from a user to determine conversational or command content, or a combination of these, the method comprising:
a. Receiving input representative of the user's intended communications;
b. Using a trained language model to separate the interpreted input into conversational components or command components, or a combination of these;
c. Generating outputs corresponding to intended conversation or command components, or a combination of these.

Clause 23: The method of Clause 22, wherein the intended communications are received from a method according to any one of Clauses 1-21.

Clause 24: The method of Clause 22, wherein the intended communications are received from acoustic speech.

Clause 25: The method of Clause 22, wherein the conversation components are intended for human or artificial intelligence interactions or a combination of these, and the command components are intended for interfacing with one or more digital technologies.

Clause 26: The method of Clause 22, wherein the generated outputs corresponding to conversation components are converted into synthesized speech or text.

Clause 27: The method of Clause 22, wherein the generated outputs corresponding to command components are converted into commands, instructions, or code for a digital technology.

Clause 28: The method of Clause 22, wherein the method is used to provide user interaction with a digital technology, including but not limited to: electronic devices, software applications, firmware applications, artificial intelligence systems or agents, embedded systems, Internet of Things (IoT) devices, cloud-based services, or the internet.

Clause 29: The method of Clause 22, wherein the method provides an intuitive human-digital technology interface that enables the user to interact with the digital technology as if communicating with a human, such as through natural language.

### Example Embodiment 2

Clause 30: A system for interpreting intended communications from a subject, the system comprising:
a. One or more sensors configured to convert physical changes of thought-expressing structures (CES) into electrical signals;
b. A processing unit configured to transform these electrical signals into data representative of intended communications;
c. An interface configured to utilize this data to perform actions or generate output that corresponds to the intended communications.

System Clause 31: The system of Clause 30, wherein the one or more sensors comprise a piezoelectric or piezoresistive sensor.

System Clause 32: The system of Clause 31, wherein the configurations of said sensors are tuneable to extract specific features from the CES.

System Clause 33: The system of Clause 30, wherein the one or more sensors comprise an optical sensor.

System Clause 34: The system of Clause 33, wherein the optical sensors are configured to extract distance information from a reference point to one or more CES.

System Clause 35: The system of Clause 30, wherein the one or more sensors are integrated into a module housing, scaffolding, or fabric that are in direct or indirect contact with the CES.

System Clause 36: The system of Clause 30, wherein the one or more sensors are integrated into a module housing, scaffolding, or fabric that are in close proximity, but not in contact with the CES.

System Clause 37: The system of Clause 30, wherein the one or more sensors comprise one or more of the following: an electrode for capturing biopotentials, strain gauge sensor, load cells, force-sensitive resistors, force transducer, capacitive sensor, resistive sensor, inductive sensor, magnetoresistive sensor, or acoustic sensors.

System Clause 38: The system of Clause 30, wherein the CES are selected from a group consisting of facial muscles, or articulatory organs, or body surface areas, or any combination of these.

System Clause 39: The system of Clause 30, wherein the processing unit is configured to apply machine learning algorithms to the electrical signals.

System Clause 40: The system of Clause 39, wherein the machine learning algorithms include neural networks.

System Clause 41: The system of Clause 30, wherein the processing unit is configured to apply language models to the electrical signals.

System Clause 42: The system of Clause 30, wherein the processing unit is configured to apply speech synthesis.

System Clause 43: The system of Clause 30, further comprising a filter for differentiating between intended communication-derived signals and other signals.

System Clause 44: A system for processing intended communications from a user to determine conversational or command content, or the combination of conversational and command content, the system comprising:
a. An input module configured to receive input representative of the user's intended communications;
b. A language model trained to separate the interpreted input into conversation components and command components;
c. An output module configured to generate outputs corresponding to the separated conversation and command components.

System Clause 45: The system of Clause 44, wherein the input module is configured to receive intended communications from a method according to any one of Clauses 1-29.

System Clause 46: The system of Clause 44, wherein the input module is configured to receive intended communications through an acoustic speech recognition system.

System Clause 47: The system of Clause 44, wherein the conversation components are for human or artificial intelligence interactions or a combination of these, and the command components are for interfacing with one or more digital technologies.

System Clause 48: The system of Clause 44, wherein the output module is configured to convert the conversation components into synthesized speech or text.

System Clause 49: The system of Clause 44, wherein the output module is configured to convert the command components into commands, instructions, or code for a digital technology.

System Clause 50: The system of Clause 44, wherein the system is configured to provide user interaction with digital technology, including but not limited to: electronic devices, software applications, or internet.

System Clause 51: The system of Clause 44, wherein the system is configured to provide an intuitive human-device interface that enables the user to communicate with the device as if the device is a human recipient of the instruction.

### Example Embodiment 3

Clause 52: An apparatus for interpreting intended communications from a subject, the apparatus comprising:
a. One or more sensors designed to convert physical changes of thought-expressing structures (CES) into electrical signals;
b. A processor that is programmed to convert these electrical signals into data representative of intended communications;
c. An output interface that uses this data to perform actions or generate output that corresponds to the intended communications.

Device Clause 53: The apparatus of Clause 52, wherein the one or more sensors include a piezoelectric or piezoresistive sensor.

Device Clause 54: The apparatus of Clause 53, wherein the configurations of said sensors are tuneable to extract specific features from the CES.

Device Clause 55: The apparatus of Clause 52, wherein the one or more sensors include an optical sensor.

Device Clause 56: The apparatus of Clause 55, wherein the optical sensors are designed to extract distance information from a reference point to one or more CES.

Device Clause 57: The apparatus of Clause 52, wherein the one or more sensors are integrated into a module housing, scaffolding, or fabric that are in direct or indirect contact with the CES.

Device Clause 58: The apparatus of Clause 52, wherein the one or more sensors are integrated into a module, housing, scaffolding, or fabric that are in close proximity, but not in contact with the CES.

Device Clause 59: The apparatus of Clause 52, whereby the apparatus includes a support structure, retention component, and anchor component to hold the apparatus in position on the CES, where the retention component may contain electrical conductive components.

Device Clause 60: The apparatus of Clause 59, whereby the retention component has a telescopic part that may be collapsable, or extendable, or auto assembling, or adjustable or a combination of these.

Device Clause 61: The apparatus of Clause 59, whereby the retention component connects to the support structure and the anchoring component with an articulation that may have zero or more degrees of freedom.

Device Clause 62: The apparatus of Clause 52, wherein the one or more sensors include one or more of the following: an electrode for capturing biopotentials, strain gauge sensor, load cells, force-sensitive resistors, force transducer, capacitive sensor, resistive sensor, inductive sensor, magnetoresistive sensor, or acoustic sensors.

Device Clause 63: The apparatus of Clause 52, wherein the CES are selected from a group consisting of facial muscles, or articulatory organs, or body surface areas, or any combination of these.

Device Clause 64: The apparatus of Clause 52, wherein the processor is programmed to process the electrical signals using machine learning algorithms.

Device Clause 65: The apparatus of Clause 64, wherein the machine learning algorithms include neural networks.

Device Clause 66: The apparatus of Clause 52, wherein the processor is programmed to process the electrical signals using language models.

Device Clause 67: The apparatus of Clause 52, wherein the processor is programmed to process the electrical signals to synthesised speech.

Device Clause 68: The apparatus of Clause 52, further comprising a filter designed to differentiate between intended communication-derived signals and other signals.

Device Clause 69: An apparatus for processing intended communications from a user to determine conversational or command content, or the combination of conversational and command content, the apparatus comprising:
a. An input module designed to receive input representative of the user's intended communications;
b. A processor programmed with a trained language model to separate the interpreted input into conversation components and command components;
c. An output module designed to generate outputs corresponding to the separated conversation and command components.

Device Clause 70: The apparatus of Clause 69, wherein the input module is designed to receive intended communications from a method according to any one of Clauses 1-29.

Device Clause 71: The apparatus of Clause 69, wherein the input module is designed to receive intended communications through an acoustic speech recognition system.

Device Clause 72: The apparatus of Clause 69, wherein the conversation components are for human or artificial intelligence interactions or a combination of these, and the command components are for interfacing with one or more digital technologies.

Device Clause 73: The apparatus of Clause 69, wherein the output module is designed to convert the conversation components into synthesized speech or text.

Device Clause 74: The apparatus of Clause 69, wherein the output module is configured to convert the command components into commands, instructions, or code for a digital technology.

Device Clause 75: The apparatus of Clause 69, wherein the apparatus is designed to provide user interaction with digital technology, including but not limited to: electronic devices, software applications, or internet.

Device Clause 76: The apparatus of Clause 69, wherein the apparatus is designed to provide an intuitive human-device interface that enables the user to communicate with the device as if the device is a human recipient of the instruction.

### Example Embodiment 4

Clause 77: A method for extracting intended communications from a subject, the method comprising:
a. Collecting electrical signals from thought-expressing structures (CES) using one or more sensors;
b. Processing these electrical signals into data representative of intended communications;
d. Utilizing this data to perform actions or generate output that corresponds to the intended communications.

Device Clause 78: The method of Clause 77, where the sensors and conducting pathways are located under the surface of the skin.

Device Clause 78: The method of Clause 77-78, where the sensors and conducting pathways are made of a conductive material, ink or wire.

### Example Embodiment 5

Clause 79: A method, system and device for extracting intended communications from a subject, which combines one or more of the sensor approaches in any of the Clauses 1-78, such as mechanical, optical and below skin acquired electrical signals.

### Example Embodiment 6

Clause 1. A speech decoding system comprising: a tattoo-based EMG sensor configured to detect electrical signals generated by a user's speech articulatory organs.

Clause 2. The system of Clause 1, wherein the tattoo-based EMG sensor is made of bio-compatible, conductive ink.

Clause 3. The system of Clause 1, wherein the tattoo-based EMG sensor is configured to dynamically track electrical signals generated by the movement of the user's speech articulatory organs in real-time.

Clause 4. The system of Clause 1, wherein the tattoo-based EMG sensor is integrated with a processing unit programmed to decode speech based on the detected electrical signals.

Clause 5. A speech decoding method comprising: using a tattoo-based EMG sensor to detect electrical signals generated by a user's speech articulatory organs; and decoding the speech based on the detected electrical signals.

Clause 6. The method of Clause 5, wherein the step of decoding the speech includes using a machine learning algorithm trained on the detected electrical signals.

Clause 7. The method of Clause 5, further including the step of calibrating the tattoo-based EMG sensor based on the individual user's speech patterns.

Clause 8. A tattoo-based EMG sensor for speech decoding, comprising: a bio-compatible, conductive ink configured to detect electrical signals generated by a user's speech articulatory organs.

Clause 9. The tattoo-based EMG sensor of Clause 8, further including a flexible substrate for adhering to the user's skin.

Clause 10. The tattoo-based EMG sensor of Clause 8, wherein the conductive ink is configured to maintain a stable contact with the user's skin to enhance signal detection.

Clause 11. The tattoo-based EMG sensor of Clause 8, wherein the conductive ink is arranged in a pattern configured to optimize the detection of electrical signals generated by the user's speech articulatory organs.

1.-18. (canceled)

19. A speech-interface system, the system comprising:
a proximal articulator module configured to be located at or adjacent to one or more speech articulators of a user and comprising an articulator-proximate sensing assembly arranged to obtain articulator-derived signals from one or more communication-expressing structures comprising at least one of facial, perioral, mandibular, craniofacial or oral structures of the user;
a base module communicatively coupled to the proximal articulator module and comprising at least one processor and a communication interface; and
a communication link between the proximal articulator module and the base module;
wherein the articulator-proximate sensing assembly comprises one or more articulator sensors configured to detect activity and/or movements of the one or more speech articulators corresponding to linguistic articulatory expressions, and to output the articulator-derived signals via the communication link to the base module;
and wherein the at least one processor of the base module is configured to process, or to cause one or more remote computing resources communicatively coupled to the base module to process, the articulator-derived signals to generate linguistic output representing an utterance of the user and to provide the linguistic output to an output device for presentation as human-perceptible communication.

20. The system of claim 19, wherein any one or more of the following conditions apply:
(a) the one or more articulator sensors comprise:
at least one of: biomechanical deformation sensors, piezoelectric sensors, strain sensors, capacitive sensors, depth-sensing components, and optical sensors configured to detect movement and/or deformation of the one or more speech articulators, and
one or more electromyographic sensors configured to sense muscle activity associated with the one or more speech articulators;
(b) the proximal articulator module and the base module cooperate such that the proximal articulator module performs signal acquisition and the base module performs linguistic decoding of the articulator-derived signals using the at least one processor; and/or
(c) the at least one processor is configured to map patterns in the articulator-derived signals to linguistic units comprising at least one of phonemes, visemes, syllables, words, phrases or sentences and to assemble the linguistic units into the linguistic output.

21. The system of claim 19, wherein the at least one processor is configured to generate one or more of:
a communication component comprising text or speech content intended to be conveyed to a recipient, and
a command component representing a control intent associated with the communication component,
and to cause the output device to present the communication component as text and/or synthesized speech while using the command component to control one or more devices or applications.

22. The system of claim 19, wherein the proximal articulator module comprises an anatomically calibrated, adjustable mounting structure shaped or adjustable to conform to a craniofacial and/or perioral contour of the user so as to maintain a predetermined spatial relationship between the articulator-proximate sensing assembly and the one or more speech articulators.

23. The system of claim 22, wherein the anatomically calibrated, adjustable mounting structure comprises at least one of:
a perioral frame, scaffold or arm configured to surround the oral cavity of the user,
a chin strap configured to extend beneath a mandible of the user,
a mask or oral appliance configured to contact intra-oral structures,
and wherein the positions of the one or more articulator sensors on the mounting structure are determined by a calibration process that aligns the articulator sensors with corresponding articulators of the user.

24. The system of claim 19, further comprising one or more subdermal conductive pathways permanently integrated beneath the skin of the user and configured to electrically couple the proximal articulator module and the base module.

25. The system of claim 24, wherein the one or more subdermal conductive pathways are implanted to follow an anatomical trajectory between a region adjacent the oral cavity of the user and a region adjacent a base module location so as to preserve a stable electrical connection for the articulator-derived signals during movement of the user; and
optionally, wherein the base module is configured to be removably coupled to a subdermal connector associated with the one or more subdermal conductive pathways so that the base module can be detached while leaving the subdermal conductive pathways and the proximal articulator module in place.

26. The system of claim 19, wherein the articulator-derived signals are obtained during sub-audible or silent speech expressions performed without generating airborne acoustic signals from vibrating vocal folds.

27. The system of claim 19, wherein the system is configured for use by a user having impaired or absent vocal fold function, and the linguistic output provides a substitute voice or text-based communication channel for the user.

28. The system of claim 19, wherein the output device comprises a speech synthesis module configured to transform the linguistic output into synthesized speech audio, and optionally a display configured to render the linguistic output as text.

29. The system of claim 19,
wherein the one or more articulator sensors comprise articulator sensors of at least two different sensing modalities selected from biomechanical deformation sensing, depth sensing and electromyographic sensing,
and wherein the at least one processor is configured to perform sensor fusion of articulator-derived signals obtained from the at least two different sensing modalities when generating the linguistic output.

30. A speech-interface system, comprising:
a sensing assembly configured to be positioned at or adjacent to one or more speech articulators of a user, the sensing assembly comprising one or more depth-sensing components, each depth-sensing component being selected from a time-of-flight depth sensor or sensor array, a LiDAR sensor or sensor array, a structured-light depth camera, an infrared depth camera, a stereo depth camera, a thermal depth camera, and combinations thereof, wherein the one or more depth-sensing components comprise one or more depth sensors, depth sensor arrays, or multiple spatially distributed depth sensor arrays;
wherein the sensing assembly is arranged such that a field-of-view of the one or more depth-sensing components extends to one or more of the intra-oral articulators within an oral cavity of the user and perioral articulators external to the oral cavity, and is configured to generate depth data representing distances between the one or more depth-sensing components and one or more of the speech articulators during linguistic articulatory expressions;
at least one processor communicatively coupled to the sensing assembly and configured to process the depth data to decode linguistic articulatory expressions of the user and to generate linguistic output corresponding to an intended utterance of the user;
wherein the linguistic articulatory expressions comprise silent or sub-audible speech performed without reliance on airborne acoustic signals generated by vibrating vocal folds.

31. The speech-interface system of claim 30,
wherein the sensing assembly comprises a mounting structure configured to support the one or more depth-sensing components with an adjustable orientation relative to the one or more speech articulators, and
wherein the adjustable orientation is set during a calibration procedure so that a field of view of the one or more depth-sensing components extends into an oral cavity of the user and covers intra-oral and/or perioral speech articulators including at least a tongue and/or lips during the linguistic articulatory expressions.

32. A proximal articulator module for a speech-interface platform, comprising:
an anatomically conforming, adjustable mounting structure configured to be worn on or supported by a head or face region of a user and to support a sensor assembly,
wherein the sensor assembly is positioned at or adjacent to one or more speech articulators of the user via one or more support arms, extensions, or intermediate support structures,
and is arranged to maintain a defined spatial registration with one or more of facial, perioral, mandibular, craniofacial or intra-oral structures;
an articulator-proximate sensing assembly supported by, or electrically coupled via, the mounting structure and comprising one or more articulator sensors or sensor arrays configured to capture, from the one or more speech articulators, at least one of mechanical movements or electrical signals associated with muscle activity, and configured to detect articulatory movements associated with linguistic articulatory expressions, and, in response, to generate articulator-derived signals representing the articulatory movements; and
a module interface configured to convey articulator-derived signals generated by one or more articulator sensors or sensor arrays to a local or remote base module for processing.

33. The proximal articulator module of claim 32, wherein the anatomically conforming, adjustable mounting structure comprises a resilient frame configured to be supported on a head or face region of the user, and one or more sensor support arms or extensions extending from the frame towards the lips, cheeks, or oral cavity region of the user, each sensor support arm carrying at least one of the articulator sensors or sensor arrays.

34. A speech-interface system comprising:
one or more subdermal articulator sensors implanted at or adjacent to one or more speech articulators of a user and configured to capture electrical signals associated with muscle activity during linguistic articulatory expressions of the user;
at least one subdermal conductive pathway implanted beneath skin of the user and electrically connected to the one or more subdermal articulator sensors and to one or more subdermal presenting electrodes located beneath skin adjacent a mounting location for a base module;
a base module comprising at least one processor and an electrical coupling interface including one or more external electrodes configured, in use, to be positioned on skin of the user adjacent the one or more subdermal presenting electrodes so as to receive articulator-derived signals via biopotentials measured across the external electrodes and the subdermal presenting electrodes; and
wherein the at least one processor of the base module is configured to process, or to cause one or more remote computing resources communicatively coupled to the base module to process, the articulator-derived signals to generate linguistic output representing an utterance of the user and to provide the linguistic output to an output device for presentation as human-perceptible communication.

35. A computer-implemented method of generating linguistic output from articulator-derived signals, the method comprising:
obtaining, by an articulator-proximate sensing assembly of a proximal articulator module worn at or adjacent to one or more speech articulators of a user, articulator-derived signals indicative of activity or movements of the one or more speech articulators corresponding to linguistic articulatory expressions;
communicating the articulator-derived signals from the proximal articulator module to a base module comprising at least one processor via a communication link that includes at least one of a wired connection and a wireless connection;
processing, by the at least one processor, the articulator-derived signals to decode linguistic units representing an intended utterance of the user and to generate linguistic output based on the linguistic units; and
causing an output device to present human-perceptible communication based on the linguistic output.

36. The method of claim 35, wherein obtaining the articulator-derived signals comprises sensing sub-audible or silent speech expressions of the user performed without generating airborne acoustic signals from vibrating vocal folds.

37. The method of claim 35, comprising one or more of:
supplying the linguistic output to a processor which determines one or more actions based on the linguistic output and causes the one or more actions to be performed, the actions comprising at least one of: rendering synthesized speech, sending a message, querying an information service, or controlling an external device or software application; and
performing sensor fusion of data obtained from one or more sensing modalities selected from biomechanical deformation sensing, depth sensing and electromyographic sensing, including fusion of multiple data channels within a single modality.

38. A non-transitory computer-readable medium storing instructions which, when executed by at least one processor of a base module of the speech-interface system, cause the at least one processor to perform the method of claim 35.
